(19) United States
(12) Patent Application Publication
(10) Pub. No.: US 2021/0116386 A1
(43) Pub. Date: Apr. 22, 2021

(54) SYSTEMS AND METHODS FOR TESTING A TEST SAMPLE

(71) Applicant: Validere Technologies Inc., Toronto (CA)

(72) Inventors: Ofer Vitner, Toronto (CA); Ian Burgess, Toronto (CA); Nouman Ahmad, Toronto (CA); John Nguyen, Toronto (CA)

(21) Appl. No.: 17/251,761

(22) PCT Filed: Jun. 11, 2019
(86) PCT No.: PCT/CA2019/050826

§ 371 Date: Dec. 11, 2020

Related U.S. Application Data
(60) Provisional application No. 62/683,623, filed on Jun. 11, 2018.
(60) Provisional application No. 62/683,625, filed on Jun. 11, 2018.

(51) Int. Cl. G01N 21/85

(52) U.S. Cl. CPC G01N 21/85; G01N 2021/0125

(57) ABSTRACT

Systems and methods for testing a test sample obtain a first image of the test sample via a first input device. The first input device is a primary camera configured to capture the first image while a plurality of light sources illuminate the test sample. The first image is sent from the first input device to a control panel. The control panel is used to label a plurality of layers on the first image. A water cut of the test sample is determined based on labeling of plurality of layers of the first image.

### CROSS-REFERENCE TO RELATED APPLICATIONS

This application claims the benefit of U.S. Provisional Application No. 62/683,623, filed Jun. 11, 2018 and U.S. Provisional Application No. 62/683,625, filed Jun. 11, 2018, which are hereby incorporated by reference herein.

### BACKGROUND

Testing for liquid samples (e.g., hydrocarbons) can reveal the contents and quality of the samples. Machines and systems that perform liquid testing are prone to error from the placement of samples within the machine, from poor lighting, and from a lack of analysis capability.

### FIELD OF THE INVENTION

The present disclosure relates generally to testing methods, computer readable mediums, and systems, and more specifically, to methods, systems, and computer readable mediums for testing test samples.

### SUMMARY

In general, in one aspect, one or more embodiments relate to a method for testing a test sample. A first image of the test sample is obtained via a first input device. The first input device is a primary camera configured to capture the first image while a plurality of light sources illuminate the test sample. The first image is sent from the first input device to a control panel. The control panel labels a plurality of layers on the first image. A water cut of the test sample is determined based on labeling of plurality of layers of the first image.

In general, in one aspect, one or more embodiments relate to a method of testing. A set of timing data is received. A set of measurement data is received. A set of analyzer data is received. A set of outputs is generated from the timing data, the measurement data, and the analyzer data. An alert is generated from an output of the set of outputs. The output and the alert are sent to a client device.

In general, in one aspect, one or more embodiments relate to a system of testing that includes a computer processor and a memory with a set of instructions that are executed by the computer processor. A first image of the test sample is obtained via a first input device. The first input device is a primary camera configured to capture the first image while a plurality of light sources illuminate the test sample. The first image from the first input device is sent to a control panel. The control panel labels a plurality of layers on the first image. A water cut of the test sample is determined based on labeling of plurality of layers of the first image.

In general, in one aspect, one or more embodiments relate to a non-transitory computer readable medium that comprises computer readable program code. The non-transitory computer readable medium comprises computer readable program code for receiving a set of timing data. The non-transitory computer readable medium comprises computer readable program code for receiving a set of measurement data. The non-transitory computer readable medium comprises computer readable program code for receiving a set of analyzer data. The non-transitory computer readable medium comprises computer readable program code for generating a set of outputs from the timing data, the measurement data, and the analyzer data. The non-transitory computer readable medium comprises computer readable program code for generating an alert from an output of the set of outputs. The non-transitory computer readable medium comprises computer readable program code for sending the output and the alert to a client device.

### BRIEF DESCRIPTION OF DRAWINGS

The present embodiments are illustrated by way of example and are not intended to be limited by the figures of the accompanying drawings.

FIGS. 1A and 1B show an example of a testing apparatus in isometric and side view, respectively, in accordance with one or more embodiments.

FIGS. 2A and 2B show an example of a testing apparatus in isometric and side view, respectively, in accordance with one or more embodiments.

FIGS. 3A and 3B show an example of a testing apparatus in isometric view in accordance with one or more embodiments.

FIG. 4 shows a side view is shown of a testing apparatus similar to FIGS. 3A and 3B in accordance with one or more embodiments.

FIG. 5 shows top view example of a testing apparatus similar to FIGS. 3A and 3B in accordance with one or more embodiments.

FIG. 6 shows an example of a testing apparatus in an isometric view in accordance with one or more embodiments.

FIG. 7 shows a top view of a testing apparatus similar to FIG. 6 in accordance with one or more embodiments.

FIG. 8 shows an exploded view of the testing apparatus from FIG. 6 in accordance with one or more embodiments.

FIGS. 9A and 9B show an isometric and top view, respectively, of one side of the testing apparatus in accordance with one or more embodiments.

FIG. 10 shows a section view of FIG. 6 taken from the inside of the testing apparatus in accordance with one or more embodiments.

FIG. 11 shows an example of a top view of a testing apparatus in accordance with one or more embodiments.

FIG. 12 shows an example of a top view of a testing apparatus in accordance with one or more embodiments.

FIG. 13 shows a flow diagram of a system for testing in accordance with one or more embodiments.

FIG. 14 shows a flowchart describing a method for capturing an image in accordance with one or more embodiments.

FIG. 15 shows a flowchart describing a method for determining analyzing a sample.

FIG. 16 shows a flowchart describing a method for capturing images.

FIG. 17 shows a flowchart describing a method for analyzing layers of a sample.

FIGS. 18A and 18B show a flowchart describing a method for determining water volume percentage in a test sample.

FIG. 19 shows a system in accordance with one or more embodiments of the present disclosure.

FIG. 20 shows a flowchart describing a method for analyzing samples.

FIG. 21 shows a diagram of a method for generating process recommendations.

FIG. 22 shows a diagram of a method for generating error probabilities.

FIG. 23 shows a flowchart of a method for generating a failure mode analysis.

FIG. 24 shows a chart used in failure mode analysis.

FIG. 25 shows a chart used in failure mode analysis.

FIG. 26 shows a chart used in failure mode analysis.

FIG. 27 shows a set of charts used in failure mode analysis.

FIGS. 28A and 28B show a computing system in accordance with one or more embodiments of the invention.

### DETAILED DESCRIPTION

The following detailed description is merely exemplary in nature, and is not intended to limit the disclosed technology or the application and uses of the disclosed technology. Furthermore, there is no intention to be bound by any expressed or implied theory presented in the preceding technical field, background, or the following detailed description.

In the following detailed description of embodiments, numerous specific details are set forth in order to provide a more thorough understanding of the disclosed technology. However, it will be apparent to one of ordinary skill in the art that the disclosed technology may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

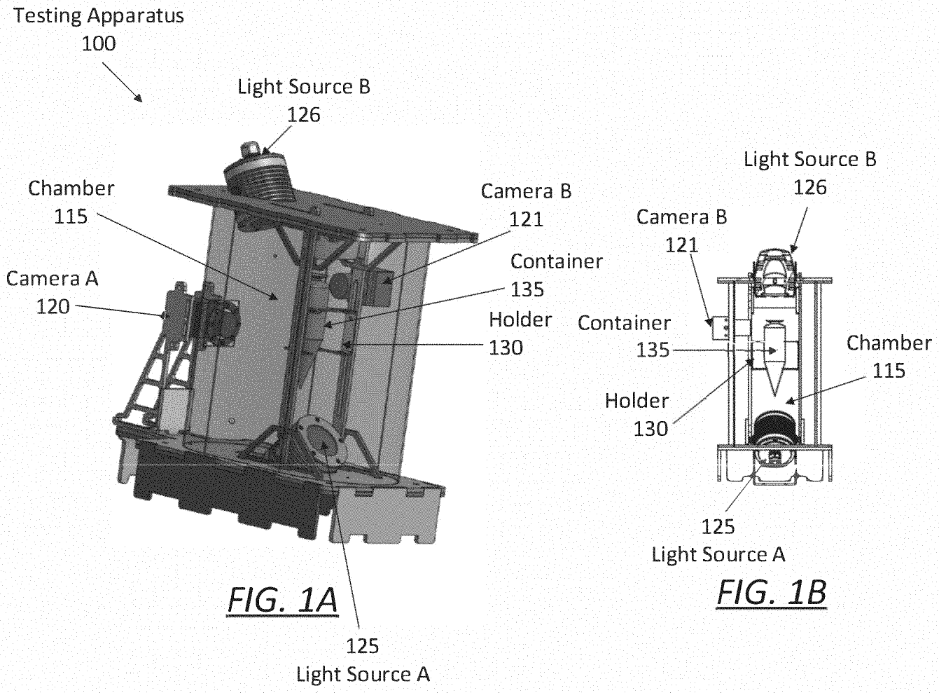

Turning now to the figures, FIGS. 1A and 1B show different views of a testing apparatus (100) in accordance with one or more embodiments of the invention. FIG. 1A shows an isometric view of an embodiment that includes one or more chambers (e.g., chamber (115)), which are areas where the various components are located. In one or more embodiments, the various components include the input devices (e.g., camera A (120), camera B (121), etc.), the container (135) that is commonly shaped as a tube that is suspended in the chamber (115) by a holder (130), and various different light sources (e.g., light source A (125), light source B (126), etc.). In one or more embodiments, one or more chambers (e.g., chamber (115)), are bounded by four walls encompassing an enclosed space. The arrangement of the components within the chamber depends heavily on the manner and type of testing performed by the testing apparatus (100).

FIG. 1B shows a side view from the position of camera A of an embodiment of the invention that includes a chamber (115) within which each of the component are at least partially contained. In one or more embodiments, the various components include the input devices (e.g., camera B (121), etc.), the container (135) that is commonly shaped as a tube that is suspended in the chamber (115) by a holder (130), and a light source (e.g., light source A (125), etc.).

Each of the components are described below.

The input devices, such as camera A (120) and camera B (121) may be integrated camera(s) as part of a tablet-based control panel (not shown), and/or industrial camera(s) depending on the particular need for the testing apparatus (100). In one or more embodiments, camera A (120) and camera B (121) may be placed within the testing apparatus (100) at a position 90 degrees with respect to each other, in order to gather more information from the different angle. Camera A (120) and camera B (121) may also be positioned at any angle with respect to each other. Camera A (120) and camera B (121) may also be at different heights within the chamber (115) to focus on different areas of interest.

In one or more embodiments, the container (135) (or tube) may be either long/short, per ASTM D4007 and/or D0097. In one or more embodiments, the position of the calibrated volume marks found on the container (135) are in front of a primary camera (e.g., camera A (120)). The holder (130) within the chamber (115) may be able to adjust to host either of the long/short containers. In one or more embodiments of the invention, the testing apparatus (100) is capable of acquiring an image of a pair of containers at a time, so two holders may be necessary.

In one or more embodiments, various different light sources (e.g., light source A (125), light source B (126), etc.) may be of any type now known or developed in the future that are capable of facilitating the ability to learn and analyze as much as possible from every bit of data from one or more images pictures coming from different input devices. The intensity, color, shape, size, angle, and the ability to control light sources separately, provides the flexibility to superimpose or manipulate the images obtained by using all/part of the light sources in accordance with one or more embodiments. Because the color of the testing sample (e.g., a hydrocarbon testing sample) varies form crystal clear to “coal black”, the light source(s) reduce reflections using post processing manipulations.

In one or more embodiments, the light sources may be ultraviolet (UV) light. It is important to understand that UV light provides another layer of analytics. To a degree, using UV light allows for detecting other materials which may be defined as soluble, yet others are not. For example, the UV light source may determine additional layers such as but not limited to: trapped water in tight emulsions, paraffin waxes, asphaltenes, drilling fluids, and other contaminants or additives. The image captured using the UV light source may show an approximation of trapped water due to the differences in fluorescence of water and oil. UV image acquisition and analysis uses differences in fluorescence in all of the constituents of the testing sample relative to their appearance in visible light. Simultaneous analysis of visible light illuminated images and UV illuminated images improve constituent classification capabilities. The UV light may be positioned in any placement around the chamber (115) where a light source is feasible. In one or more embodiments, the UV light source may be the only light source in the chamber (115).

In one or more embodiments, the light sources may have different angles, heights, shapes, and types. In particular, the type of light sources may include LEDs, UV in a ‘ring’ like shape together with ‘white’ LEDs on the same ring PCB, a long bar with an array of LEDs illuminating a cylindrical chamber in order to minimize reflection and provide homogeneous light around the container without directly ‘hitting’ the container, as well as short arrays of LEDs, around the tube in different heights, illuminating the container directly, with different angles.

In one or more embodiments, the chamber (115) is a flat shape or may be a round, cylindrical shape. The round, cylindrical shape behind the container (135) improves the illumination of the container (135) when using indirect light by providing light arrays to reflect from that curved wall backwards to the ‘back’ side of the container (135). In one or more embodiments, the color painted in the chamber (115) is also of an importance, to assure good analytics of the testing sample, regardless of the sample color, under different light conditions.

One or more embodiments of the testing apparatus (100) provides one or more the following advantages or benefits. The testing apparatus (100) is capable of recording sample data for use during future disputes. The testing apparatus (100) exports the data (e.g., image(s), user data, materials added to the sample, type of sample, etc.) via WiFi/cellular communication to a secured database. The testing apparatus (100) may work in online as well as offline modes. The testing apparatus (100) may be introduced to harsh conditions. The testing apparatus (100) may be used both indoors and outdoors. The testing apparatus (100) may be portable and mounted on a vehicle per need. The testing apparatus (100) may be both corded or cordless (using a battery). The service(s) associate with the testing apparatus (100) provides value—a number in which the tester knows the quantity of different materials in that sample. The service(s) associate with the testing apparatus (100) may provide a set of analytical tools related to the suppliers of customers, consistent quality, integrity, internal quality procedures and processes, etc.

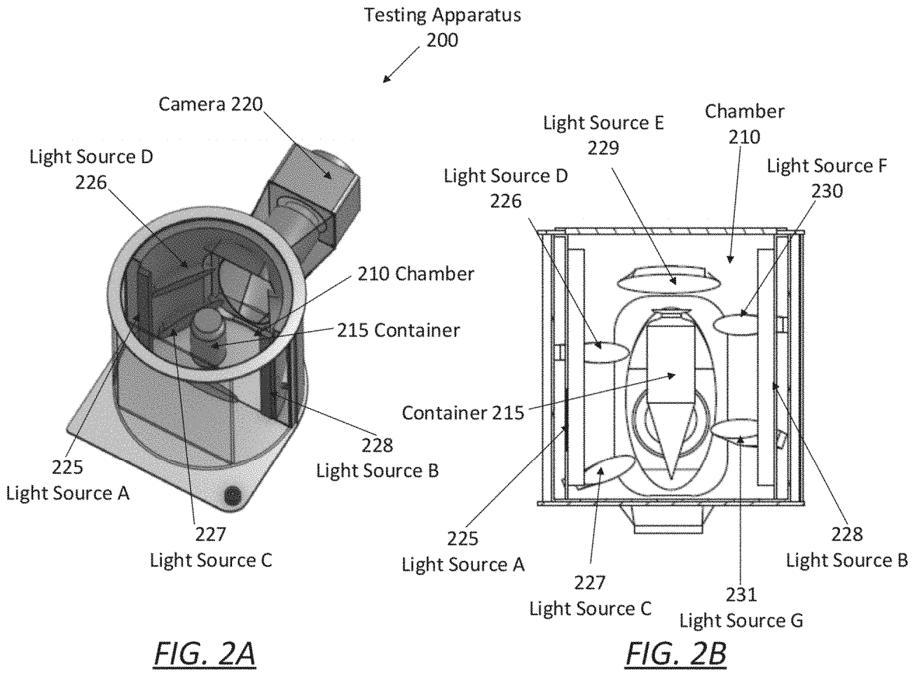

FIGS. 2A and 2B show a testing apparatus (200) in accordance with one or more embodiments of the invention. The testing apparatus (200) show a different embodiment with many of the same features shown and described in relation to FIGS. 1A and 1B. In particular FIGS. 2A and 2B show a camera (220), multiple lights sources (e.g., light source A (225), light source B (228), light source C (227), light source D (226), light source E (229), light source F (230), light source G (230)), a container (215), and a chamber (210).

Each of the components are described below.

The camera (220) may be a similar camera as described in FIG. 1. In one or more embodiments, the camera (220) is located on one end of a shaft extending from the chamber (210). The camera (220) is positioned in a manner to focus on the container (215) located in the center of the chamber (210). The container (215) contains the hydrocarbon test sample and may be labeled with volume marks to show the volume of the container (215).

In one or more embodiments, light source A (225) and light source B (228) are vertical bar lights oriented in a vertical direction along opposite walls of the chamber (210). Light source A (225) and light source B (228) may be similar to the light sources described in FIG. 1. One of ordinary skill in the art will appreciate that the placement of light source A (225) and light source B (228) in FIG. 2A differs from the placement shown in FIG. 1, however, light sources are able to illuminate the container (215) in both figures. Additionally, FIG. 2A adds a light source C (227) and light source D (226). Both light source C and light source D may be bar lights placed in a horizontal direction and perpendicular to light source A (225). In one or more embodiments, light source C (227) may be placed directly below light source D (226) at a predetermined height or any height. In one or more embodiments, the placement of light source A (225), light source B (228), light source C (227), and light source D (225) may be such that the light sources illuminate a wall within the chamber (210) that reflects onto the container (215).

FIG. 2B shows a cross section view of FIG. 2A. In addition to light source A (225), light source B (228), light source C (227), and light source D (226), FIG. 2B shows the placement of a light source E (229), a light source F (230), and a light source G (231) located within the chamber (210). In one or more embodiments, the placement of light source E (229) is located above the shaft opening into the chamber (210). Light source E (229) may be placed at an angle that allows for the light source to illuminate directly on the container (215). In one or more embodiments, light source E (229) may be placed at a different angle to allow the light source to illuminate the container (215) indirectly (i.e., reflection off a wall). In one or more embodiments, light source F (230) and light source G (231) may be light bars placed in a horizontal direction and perpendicular to light source B (228). In one or more embodiments, light source G (231) may be placed directly below light source F (230) at a predetermined height or at any height. One of ordinary skill in the art will appreciate that the placement of the light sources (e.g., light source A (225), light source B (228), light source C (227), light source D (226), light source E (229), light source F (230), light source G (230)) is not limited to the embodiment discussed, and that the light sources may be positioned in any number of ways around the chamber (210) to illuminate the container (215).

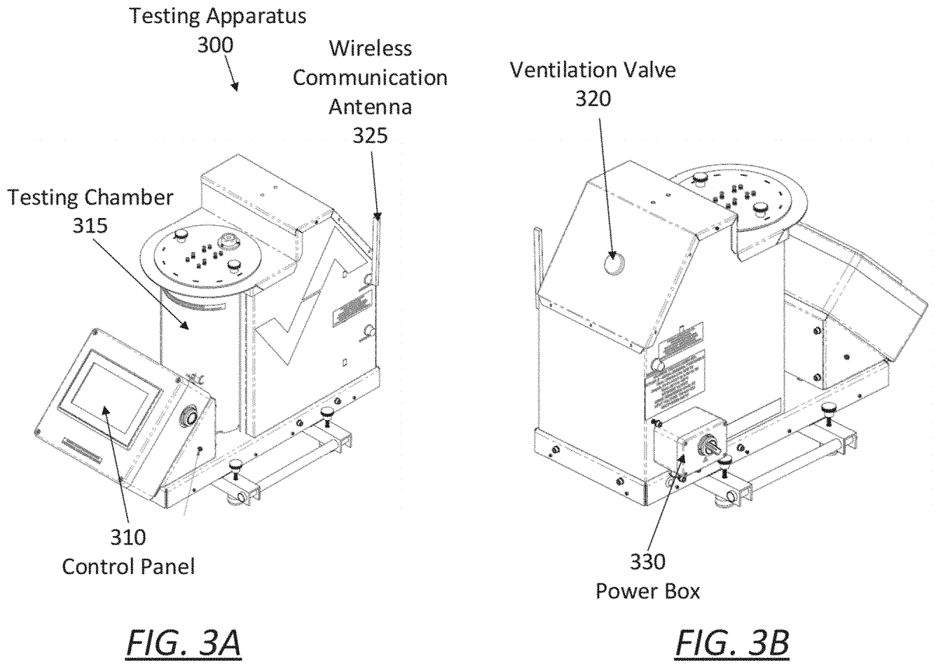

FIGS. 3A and 3B show an example of a testing apparatus (300) in isometric view. As shown in FIG. 3A, one side of the testing apparatus includes of a control panel (310) a wireless communication antenna (325), and a testing chamber (315). FIG. 3B shows the opposite side of FIG. 3A, which includes a ventilation valve (320) and a power box (330). In one or more embodiments, the testing apparatus (300) may be used to test hydrocarbons.

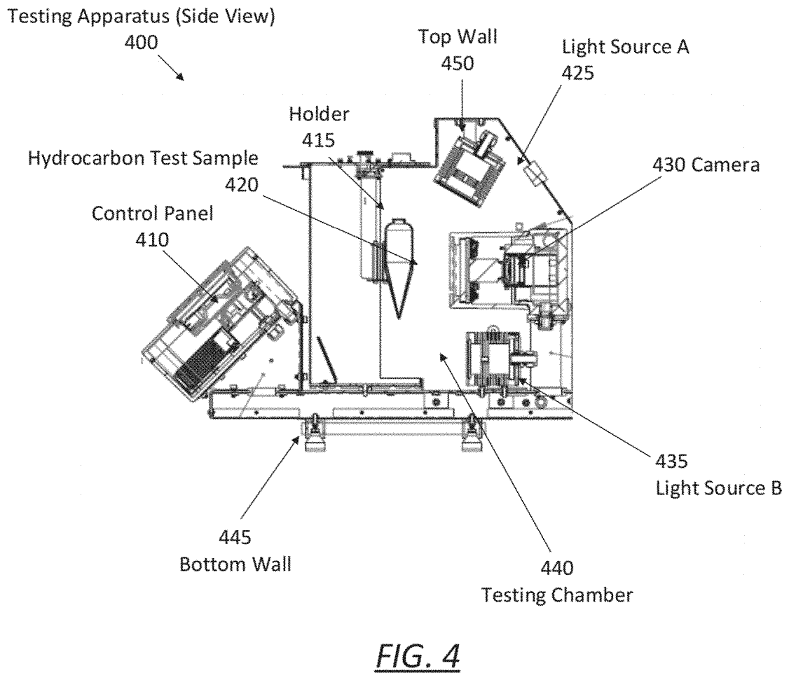

In one or more embodiments, a control panel (310) is located on the outer portion of the testing apparatus (300). The control panel (310) may be located separate from the testing chamber (315). In one or more embodiments, the control panel (310) is aligned and part of the testing chamber (315). The control panel (310) may be angled to allow a user clear visibility of the display screen. For example, prior tests may be carried out to determine that the ideal viewing angle for the control panel (310) is 45 degrees. The control panel (310) is discussed below and in relation to FIG. 4.

The testing chamber (315), in accordance with one or more embodiments, is located in the central portion of the testing apparatus (300). In one or more embodiments, the testing chamber (315) is cylindrical. The testing chamber (315) may be made of the same material as the testing apparatus (300) such as stainless steel, aluminum, copper-aluminum alloy, non-sparking metal, carbon steel, class 1 div 2 explosion proof material, iron, carbon fiber, plastic, or any other type of material. The inside of the testing chamber (315) may be a different color than the rest of the testing apparatus (300). For example, the inside of the testing chamber (315) may be painted red to allow for better viewing and operating conditions of the camera (not shown) and better illumination of the multitude of light sources (not shown). In one or more embodiments, the outer wall of the testing chamber (315) may rotate to allow access inside.

In one or more embodiments, the wireless communication antenna (325) is located on the outer portion of the testing apparatus (300). The wireless communication antenna (325) may contain capability to send data over a wireless network to another computing device (not shown). The wireless communication antenna (325) may utilize any type of wireless network (e.g., WiFi, Bluetooth, cellular network). For example, a user may wish to send data stored on the control panel (310) to another computing device. The wireless communication antenna (325) allows the user to send the data over a wireless network, such as WiFi.

In one or more testing apparatus embodiments, a ventilation valve (320) is positioned on the outer portion of the (300) as shown in FIG. 3B. The ventilation valve (320) may be opened and closed to allow air ventilation into the testing chamber or allow for gas to escape the testing chamber (315). For example, a hydrocarbon test sample may be placed inside the testing chamber (315) and the ventilation valve (320) is opened to allow for flammable gas to escape and not build up inside the testing chamber (315). The ventilation valve (320) may be made of the same material as the testing apparatus (300) such as stainless steel, aluminum, copper-aluminum alloy, non-sparking metal, carbon steel, class 1 div 2 explosion proof material, iron, carbon fiber, plastic, or any other type of material.

In one or more embodiments, a power box (330) is located on the outside of the testing apparatus (300). In one or more embodiments, the power box (330) may be located inside the testing apparatus (300). The power box (330) may include of an on/off switch, button, or any other type of mechanism to allow a user to turn the power on and off. The power box (330) may be encased in the same material as the testing apparatus (300) such as stainless steel, aluminum, copper-aluminum alloy, non-sparking metal, carbon steel, class 1 div 2 explosion proof material, iron, carbon fiber, plastic, or any other type of material.

Turning to FIG. 4, a side section view is shown of a testing apparatus (400) similar to FIGS. 3A and 3B. As shown in FIG. 4, the testing apparatus includes of a control panel (410) located on the outside and a testing chamber (440), which is separate from the control panel (410). The testing chamber (440) is defined by a top wall (450) and a bottom wall (445). A first light source (e.g., light source A (425)) is located on the top wall (450) inside of the testing chamber (440) while a second light source (e.g., light source B (435)) is located on the bottom wall (450) inside of the testing chamber (440). A holder (415) extends from the top wall (450) towards the bottom wall (445). A hydrocarbon test sample (420) is positioned in the holder (415) and a camera (430) is located opposite of the hydrocarbon test sample (420).

In one or more embodiments, the control panel (410) extends from the bottom wall (445) outside of the testing apparatus. The control panel (410) is positioned in such a manner that allows a user to view and interact with the control panel (410) while the testing apparatus (400) rests in a horizontal position. In one or more embodiments, the control panel (410) allows the user to control the camera (430) as well as the first light source (425) and the second light source (435). For example, the user may use the control panel (410) to use the camera (430) to zoom in and out, focus the camera (430) adjust the flash settings of the first light source (425) and the second light source (435), and provide input to capture an image using the camera (430) while the first light source (425) and second light source (435) illuminate the testing chamber. In one or more embodiments, the control panel (410) includes of a GPS device that allows the multitude of images to be tagged with a geographic location. For example, when an image is captured by the camera (430), the image is sent to the control panel via a network (e.g., wireless network, Bluetooth, cellular network), a connection cable (e.g., USB cable, ethernet cable, VGA cable). Or a portable storage device (e.g., USB drive, portable hard drive).

In one or more embodiments, the control panel (410) allows the user to view the multitude of images captured by the camera (430). The user may analyze the multitude of images on the control panel (410), or the control panel may analyze the multitude of images using image processing.

The control panel (410), in accordance with one or more embodiments, may identify errors in the multitude of images through the use of image processing. For example, the control panel (410) may run an algorithm to check that the layers of an image are labeled correctly. If a label is found to be incorrect, the control panel (410) may notify a user on the display screen that there is an error with the image. In one or more embodiments, the control panel (410) may identify errors in the image based on data collected from online analyzing tools. For example, the control panel (410) may detect that an image tagged with a certain geographic location has data that conflicts with data from an online analyzing tool that has a similar geographic location. An alert is sent to the display screen of the control panel (410) and a user may verify the error. In one or more embodiments, the alert is sent to a mobile device of the user or a separate computing device.

In one or more embodiments, a holder (415) extends from the top wall (450) to the center of the testing chamber (440). The holder (415) is attached to the top wall (450) via a fastening device (e.g., a screw, nut and bolt, pin). The holder (415) may be made out of the same material as the testing apparatus (400) or a different material entirely. For example, the holder (415) may be made out of stainless steel, aluminum, copper-aluminum alloy, non-sparking metal, carbon steel, class 1 div 2 explosion proof material, iron, carbon fiber, plastic, or any other type of material. The holder (415) may be of any length that allows for the hydrocarbon test sample (420) to rest opposite of the camera (430).

The hydrocarbon test sample (420), in accordance with one or more embodiments, is a container that includes of a mixture of liquids, gases, and solids. The hydrocarbon test sample (420) may be of any shape suitable for containing the mixture. For example, the hydrocarbon test sample (420) may be a test tube, a cylindrical tube, a flask, a beaker, or any other type of container holds a liquid. For example, the container may be a tube that is manufactured in accordance with ASTM D4007 or ASTM D0097. The hydrocarbon test sample (420) may be made of glass, plastic, acrylic glass, or any other type of material which allows for the user, camera (430), and control panel (410) to see the liquid, solid, and gas phases. In one or more embodiments, the hydrocarbon test sample (420) is labeled with volume marks to represent the volume of the container. The volume marks are located in a manner to be visible by the set of cameras. The volume may be indicated in liters, milliliters, cm³, fluid ounces, or any other type of measure for volume.

In one or more embodiments, the testing apparatus (400) includes of a first light source (425) and a second light source (435). The first light source (425) may be located on the top wall (450) while the second light source (435) may be located on the bottom wall (445). Both the first light source (425) and second light source (435) may be attached to the testing apparatus (400) via a fastening device (e.g., a screw, nut and bolt, pin) or a support mechanism. For example, the first light source (425) may be attached to the top wall (450) by a support rod that extends down from the top wall (450). The support rod may be fastened to both the top wall (450) and the first light source (425) by screws. However, the second light source (435) may be fastened to the bottom wall (445) with screws only. The first light source (425) and second light source (435) may be a LED strip, a LED bulb, or any other type of light source that illuminates the testing chamber. In one or more embodiments, the first light source (425) and the second light source (435) may be encased in the same material as the testing apparatus (400), such as stainless steel, aluminum, copper-aluminum alloy, non-sparking metal, carbon steel, class 1 div 2 explosion proof material, iron, carbon fiber, plastic, or any other type of material. For example, the first light source (425) and second light source (435) may both be encased in a class 1 div 2 explosion proof material.

In one or more embodiments, the camera (430) is located on a wall opposite the hydrocarbon test sample (420) and testing chamber. The camera (430) may be any type of input device capable of capturing a multitude of images. In one or more embodiments, the camera (430) is attached to the testing apparatus (400) via a mounting device. The mounting device may be attached to both the camera (430) and testing apparatus via a fastening device (e.g., a screw, nut and bolt, pin). In one or more embodiments, the camera (430) may be encased in the same material as the testing apparatus (400), such as stainless steel, aluminum, copper-aluminum alloy, non-sparking metal, carbon steel, class 1 div 2 explosion proof material, iron, carbon fiber, plastic, or any other type of material. For example, the first camera (430) may both be encased in a class 1 div 2 explosion proof material.

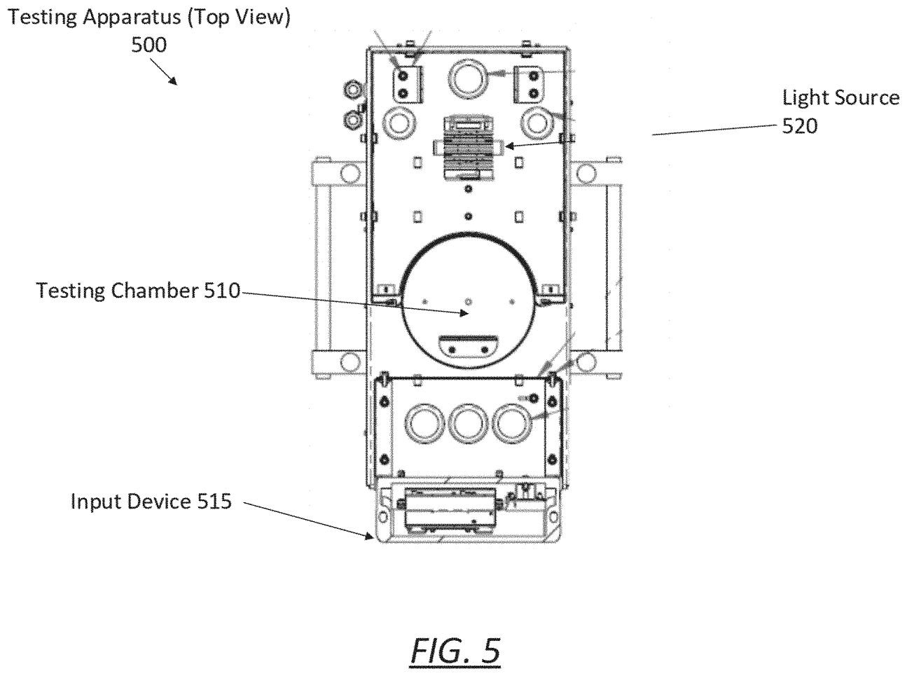

FIG. 5 shows top view example of a testing apparatus (500) similar to FIGS. 3A and 3B. As shown, the testing apparatus (500) includes of a light source (520) located inside the testing apparatus (500). The light source (520) is facing towards a testing chamber (510). A control panel (515) is located on the outer portion of the testing apparatus (500).

In one or more embodiments of the invention, the testing apparatus (500) includes of a testing chamber (510) that is centrally located. The testing chamber (510) may be shaped like a cylinder to allow for better illumination of the testing chamber (510) from the light sources (520). The testing chamber (510) may be made of the same materials as the testing apparatus (500). The testing chamber (510) may be made of stainless steel, aluminum, copper-aluminum alloy, non-sparking metal, carbon steel, class 1 div 2 explosion proof material, iron, or any other type of material. For example, the testing chamber (510) may be made out of a stainless steel rated for a class 1 div 2 hazardous area classification to ensure that the testing chamber (510) operates safely in conditions containing hazardous vapors, gases, or other flammable substances that could result in an explosion.

In one or more embodiments, a light source (520) is located on the top wall of the testing apparatus (500) inside the testing apparatus. As previously discussed, the light source (520) may be angled towards the hydrocarbon test sample. In one or more embodiments, the light source (520) is a LED light.

In one or more embodiments, a control panel (515) is located on the outside of the testing apparatus (500). The control panel (515) allows for a user to interact with the testing apparatus (500) by viewing a multitude of images captured in the testing chamber (510). In one or more embodiments, the control panel (515) allows the user to operate the camera (not shown) and light sources (520) located inside the testing apparatus (500).

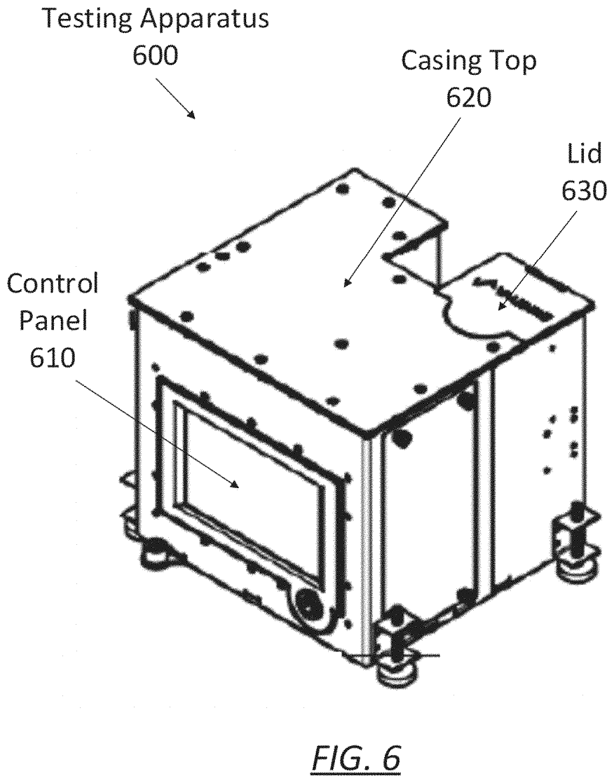

FIG. 6 shows an example of a testing apparatus (600) in an isometric view. As shown in FIG. 6, the outside of the testing apparatus (600) contains a control panel (610) located on one wall and a lid (620) located on the top portion (abutting the casing top (620)) of the testing apparatus (600).

The testing apparatus (600) is an enclosed container that allows for access into the container via the lid (630). In one or more embodiments, the testing apparatus is leak-proof and sealed to allow for the inside of the container to stay dry from outside moisture. In one or more embodiments, the testing apparatus may have openings to allow oil or any other liquid to drain out of the testing apparatus. The openings may be located in the floor or along the bottom portions of the walls. In one or more embodiments, the testing apparatus may contain a separate chamber that collects oil or liquid run-off. For example, the testing apparatus may slope towards the second chamber to allow for oil to drain into the chamber to collect. The separate chamber may be accessed at any time to remove the oil or liquid run-off. The testing apparatus (600) may be made out of stainless steel, aluminum, copper-aluminum alloy, non-sparking metal, carbon steel, class 1 div 2 explosion proof material, iron, or any other type of oil-resistant material. For example, the testing apparatus (600) may be made out of a stainless steel rated for a class 1 div 2 hazardous area classification to ensure that the testing apparatus operates safely in conditions containing hazardous vapors, gases, or other flammable substances that could result in an explosion.

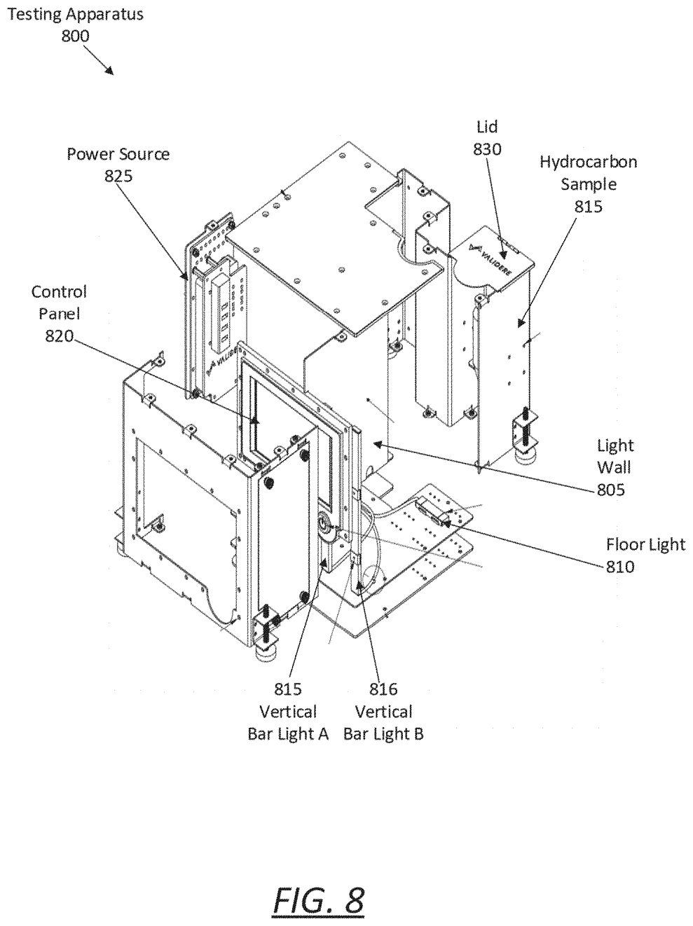

The control panel (610), in accordance with one or more embodiments, is located on the outside portion of the testing apparatus (600). The control panel (610) may be enclosed in a case which then inserts into a side wall. The control panel (600) is discussed in further detail below in FIG. 8.

In one or more embodiments of the invention, the lid (630) is located on the top portion of the testing apparatus (600). The lid (630) may open in a direction towards the user to allow for access inside the testing apparatus (600). In one or more embodiments of the invention, the lid (630) is attached to the testing apparatus (600) via a hinge. In one or more embodiments, the lid (630) is made of the same material as the testing apparatus (600).

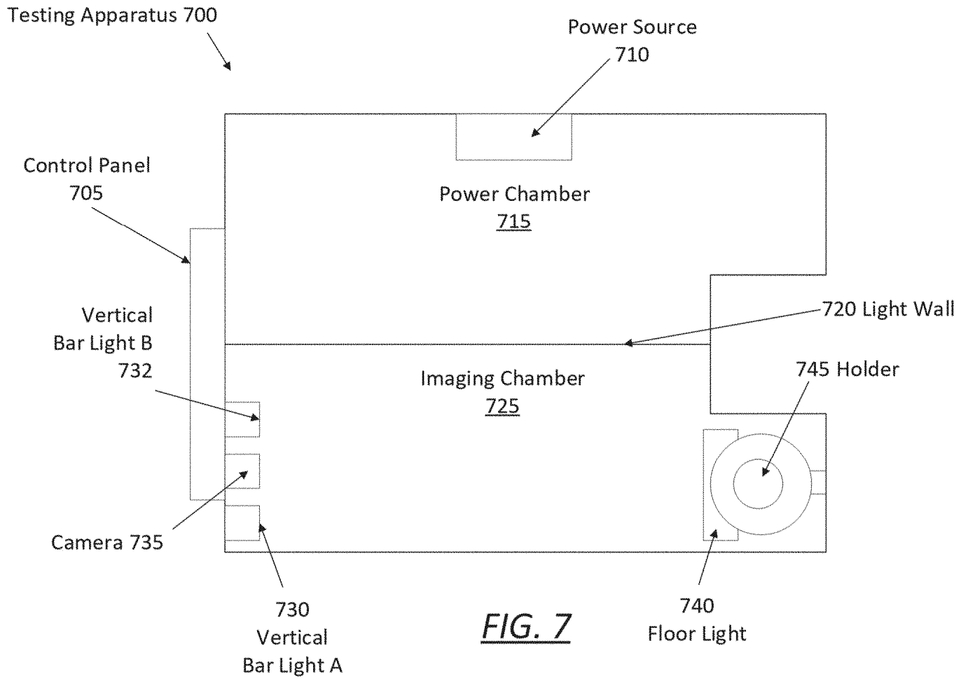

Turning to FIG. 7, a top view of a testing apparatus (700) similar to FIG. 6 is shown. As shown in the figure, the testing apparatus (700) includes of a control panel (705) located on the outside. The inside of the testing apparatus includes of two chambers, a power chamber (715) and a imaging chamber (725). The two chambers are separated by a light wall (720). A power source (710) is located on one wall of the power chamber (715). The imaging chamber (725) includes of a camera (735), vertical light bars (e.g., vertical bar light A (730), vertical bar light B (732), etc.), a floor light (740), and a holder (745).

In one or more embodiments, the control panel (705) is located on a wall on the outer portion of the testing apparatus (700). The control panel (705) may allow a user to access the data and a multitude of images captured by the camera. The control panel (705) is discussed in further detail below and in FIG. 8.

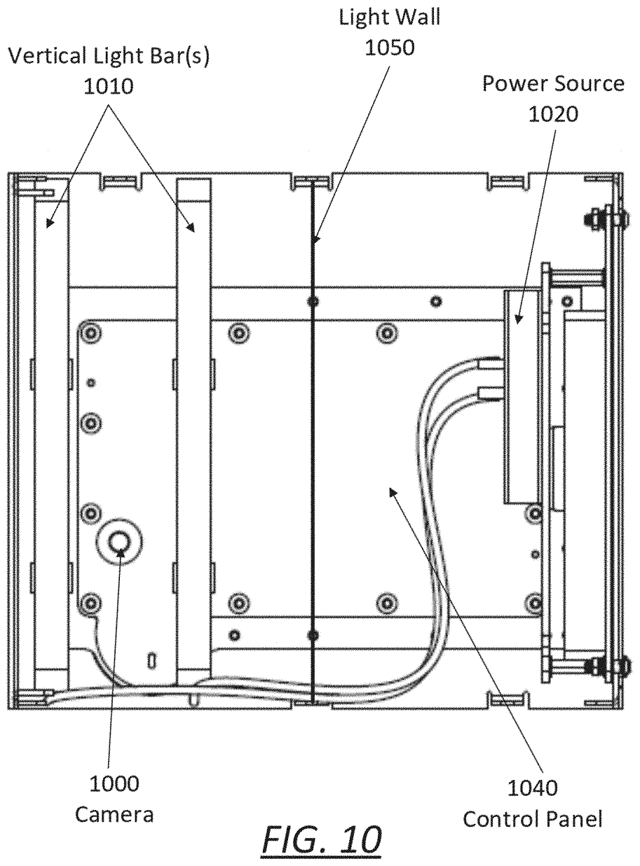

In one or more embodiments, a power source (710) is located in the power chamber (715). The power source (710) may be located on any wall inside the power chamber (715). In one or more embodiments, the power source (710) contains the capacity to store an electric charge or electric battery. In one or more embodiments, the power source (710) receives power from an outside source, such as an electrical outlet or generator, that runs the electronic devices in the testing apparatus (700). The power source (710) is discussed in further detail below and in FIG. 10.

In one or more embodiments, a light wall (720) separates the testing apparatus (700) into multiple chambers. The light wall (720) runs the length of the testing apparatus (700) from one wall to an opposite wall, sufficient to divide the testing apparatus (700) into multiple chambers. In one or more embodiments, the placement of the light wall (720) is dependent upon the light conditions. Prior tests may be done to determine the placement of the light wall (720) to provide the best illumination results for the imaging chamber (725). The light wall (720) may be made of the same material as the testing apparatus (700). In one or more embodiments, the light wall (720) is made of a reflective material to direct the light from the light sources towards a hydrocarbon test sample (not shown) positioned on the holder (745).

A camera (735) is located on a wall inside the imaging chamber (725). In one or more embodiments, the camera (735) is part of the control panel (705) and is controlled by the control panel. For example, a user may view the control panel (705) to focus and adjust the camera (735). The user may then select to capture an image of the hydrocarbon test sample (not shown) by using the control panel (705). In one or more embodiments, the camera (735) is operated separately from the control panel (705). For example, the camera (705) may be a stand-alone device placed in the imaging chamber (725) controlled remotely by a user through a user device such as a mobile device, camera control device, or any other device that may control a camera remotely. In one or more embodiments, the camera (735) may be encased in a material similar to the testing apparatus (700). For example, the camera (735) may be encased in a class 1 div 2 material that allows for the camera to be explosion proof during operation.

In one or more embodiments, the vertical light bars (e.g., vertical bar light A (730), vertical bar light B (732), etc.) are located on the same wall as the camera (735). In one or more embodiments, a vertical light bar (e.g., vertical bar light A (730), vertical bar light B (732), etc.) may be located on either side of the camera (735). The vertical light bars (e.g., vertical bar light A (730), vertical bar light B (732), etc.) are discussed below and in FIGS. 8 and 10.

In one or more embodiments, the floor light (740) is located on the floor of the testing apparatus (700) below the holder (745). In one or more embodiments, the floor light (740) is a LED strip that is the same length as the holder. The floor light (740) is discussed in further detail below and in FIGS. 8 and 10.

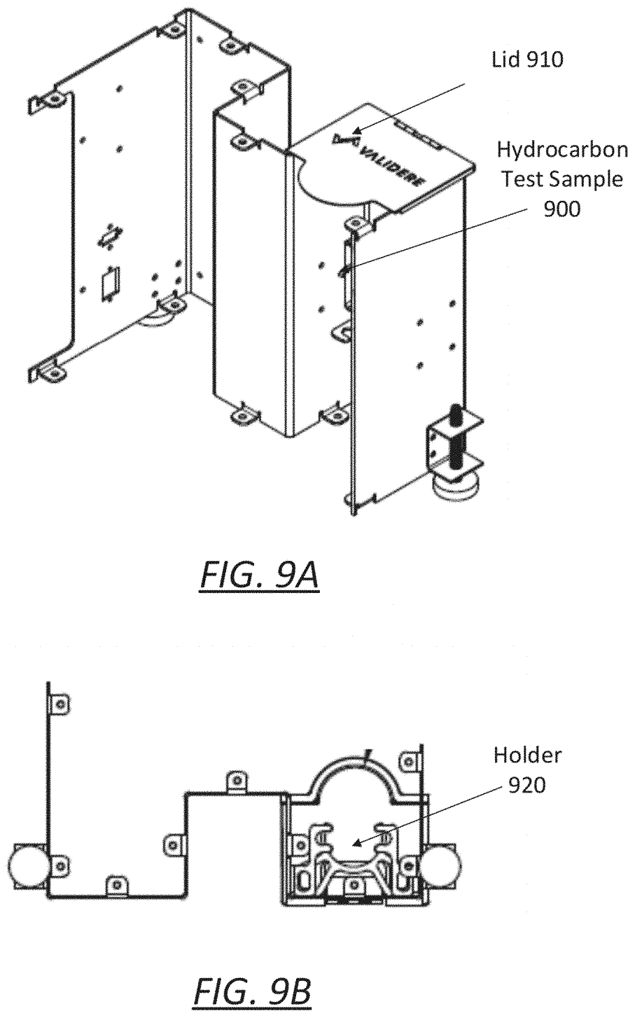

In one or more embodiments, the holder (745) is located on a wall of the testing apparatus (700) opposite of the camera (735). The holder (745) may have a circular design, rectangular design, triangular design, or any other type of design. The holder (745) may be made out of the same material as the hydrocarbon test apparatus (700). In one or more embodiments, the holder (745) is a different material than they testing apparatus (700), such as plastic, stainless steel, carbon steel, iron, or any other type of material. The holder (745) is discussed in further detail below and in FIG. 9.

FIG. 8 shows an exploded view of the testing apparatus (800) from FIG. 6. As shown in FIG. 8, the testing apparatus contains a control panel (820) and a lid (830) on the outside. A light wall (805) divides they testing apparatus (800) in separate chambers. One chamber contains a power source (825) located on a wall of the testing apparatus. The chamber on the opposite side of the light wall (805) contains vertical light bars (e.g., vertical bar light A (815), vertical bar light B (816), etc.) on the inside of the wall sharing the control panel (820). A floor light (810) is located on the floor under the hydrocarbon test sample (815), which is located on a wall opposite of the control panel.

In one or more embodiments, the control panel (820) is located on the outside portion of the testing apparatus (800). As shown in FIG. 8, the control panel (820) may be enclosed in a case which then inserts into a side wall and enclosed by a top and bottom wall. In one or more embodiments, the control panel encasement and the testing apparatus (800) are made of the same material. The material may be stainless steel, aluminum, copper-aluminum alloy, non-sparking metal, carbon steel, class 1 div 2 explosion proof material, iron, or any other type of material.

The control panel (820) may be an input device that allows for user interaction. The control panel may be a tablet computing device, a laptop, a computer, a mobile device, or any other type of computing device that allows for user interaction. In one or more embodiments, the control panel (820) is powered by the power source (825). In one or more embodiments, the control panel (820) houses the camera that captures a multitude of images of the hydrocarbon test sample (815).

As previously discussed, the vertical light bars (e.g., vertical bar light A (815), vertical bar light B (816), etc.) are located on the wall of the testing apparatus (800) housing the control panel (820). In one or more embodiments, a vertical light bar (e.g., vertical bar light A (815), vertical bar light B (816), etc.) is placed on either side of a camera (not shown) inside the testing apparatus (800). The vertical light bars (e.g., vertical bar light A (815), vertical bar light B (816), etc.) may be facing the hydrocarbon test sample (815). In one or more embodiments, the vertical light bars (e.g., vertical bar light A (815), vertical bar light B (816), etc.) is a LED strip.

The floor light (820), in accordance with one or more embodiments, is located on the floor inside of the testing apparatus (800). The floor light (820) may be located underneath the hydrocarbon test sample (815). In one or more embodiments, the floor light (820) is a LED strip that illuminates the hydrocarbon test sample (815).

In one or more embodiments, the hydrocarbon test sample (815) is located underneath a lid (830) on a wall opposite of the control panel (820). The hydrocarbon test sample (815) and the lid (830) are discussed in further detail below and in FIG. 9.

In one or more embodiments, a light wall (805) is a wall located inside the testing apparatus (800) that divides the testing apparatus into two chambers. In one or more embodiments, the power source (825) is located on one wall of the testing apparatus (800) opposite the light wall (805). The power source (825) and the light wall (805) are discussed below and in FIG. 10.

FIGS. 9A and 9B show an isometric and top view, respectively, of one side of the testing apparatus. As shown in FIG. 9A the hydrocarbon test sample (900) is located inside a testing chamber of the testing apparatus. Access to the testing chamber is provided by the lid (910) located on top of the testing apparatus. The hydrocarbon test sample (900) is positioned on a holder (920) attached to the wall of the testing chamber.

In one or more embodiments, the lid (910) is located on the top portion of the testing apparatus, as shown in FIG. 9A. The lid (910) may be attached to the testing apparatus with a hinge to allow the lid to open and close. In one or more embodiments, the lid (910) is attached to the testing apparatus with a sliding rail system that allows for the lid to slide from a closed position to an open position. In one or more embodiments, the lid (910) may be made of the same material as the testing apparatus. The lid may be made out of stainless steel, aluminum, copper-aluminum alloy, non-sparking metal, carbon steel, class 1 div 2 explosion proof material, iron, carbon fiber, plastic, or any other type of material.

In one or more embodiments, the hydrocarbon test sample (900) is located below the lid (910) to allow for access into the testing apparatus. The hydrocarbon test sample (900) may be stored in a container. The container may be a test tube, a cylindrical tube, a flask, a beaker, or any other type of container that holds the hydrocarbon test sample (900). For example, the container may be a tube that is manufactured in accordance with ASTM D4007 or ASTM D0097. The container may be made of glass, plastic, acrylic glass, or any other type of material which allows for the user or control panel to see the hydrocarbon test sample (900).

In one or more embodiments, the holder (920) is located on a wall of the apparatus under the lid (910), as shown in FIG. 9B. The holder (920) may have a circular design to support the hydrocarbon test sample (900), however, the design of the holder (920) is not limited to being circular. The holder may be rectangular, triangle, or any other type of design that supports the hydrocarbon test sample (900). The holder (920) may be made out of the same material as the test apparatus. In one or more embodiments, the holder (920) is a different material than they test apparatus, such as plastic, stainless steel, carbon steel, iron, or any other type of material.

FIG. 10 shows a section view of FIG. 6 taken from the inside of the testing apparatus and facing towards the control panel (1040). As shown in FIG. 10, the control panel (1040) includes of a camera (1000) that faces towards a hydrocarbon test sample (not shown). Vertical light bar(s) (1010) are placed on either side of the camera and are powered by a power source (1020). A light wall (1050) divides the testing apparatus into two chambers.

In one or more embodiments, the camera (1000) is built in to the control panel (1040). The camera (1000) may capture an image and the details of the image, including a time stamp and a geographic location, may all be stored internally on the control panel (1040). For example, the control panel (1040) may be a tablet computing device that includes a camera (1000) on the opposite side of the display screen of the control panel (1040). The control panel may be powered by the power source (1020), which is located in the power chamber. In one or more embodiments, the camera (1000) may be separate from the control panel (1040).

In one or more embodiments, a vertical light bar (1010) is located on both sides of the camera (1000). The vertical light bars (1010) are parallel with respect to one another and run the bottom of the testing apparatus to the top of the testing apparatus. The vertical light bars (1010) may be LED light strips or any other type of light source that illuminates the hydrocarbon testing chamber (not shown). In one or more embodiments, the vertical light bars (1010) may receive power from the power source (1020) via a wire or cable attachment. In one or more embodiments, the vertical light bars (1010) and the power source (1020) are located in separate chambers.

In one or more embodiments, the control panel (1040) is an input device that allows a user to provide input for the testing apparatus. The control panel (1040) may be located on the outside of the testing apparatus to allow the user to have direct access. The control panel (1040) may be a tablet computing device, computer, laptop, mobile device, or any other type of device that allows a user to input and receive data for the testing apparatus. For example, the control panel (1040) may be a tablet computing device attached to the testing apparatus and receives power from the power source (1020).

In one or more embodiments, the power source (1020) is located in the power chamber along a wall. The power source (1020) provides power to the testing apparatus. For example, the power source provides power to the control panel (1040) and the vertical light bar (1010) via a wire, a USB connection, or any other type of connection that transmits power from one source to another. In one or more embodiments, the power source (1020) may be a USB power supply that stores an electrical charge. For example, the power source may be used out in the field or a location that does not have access to a power outlet.

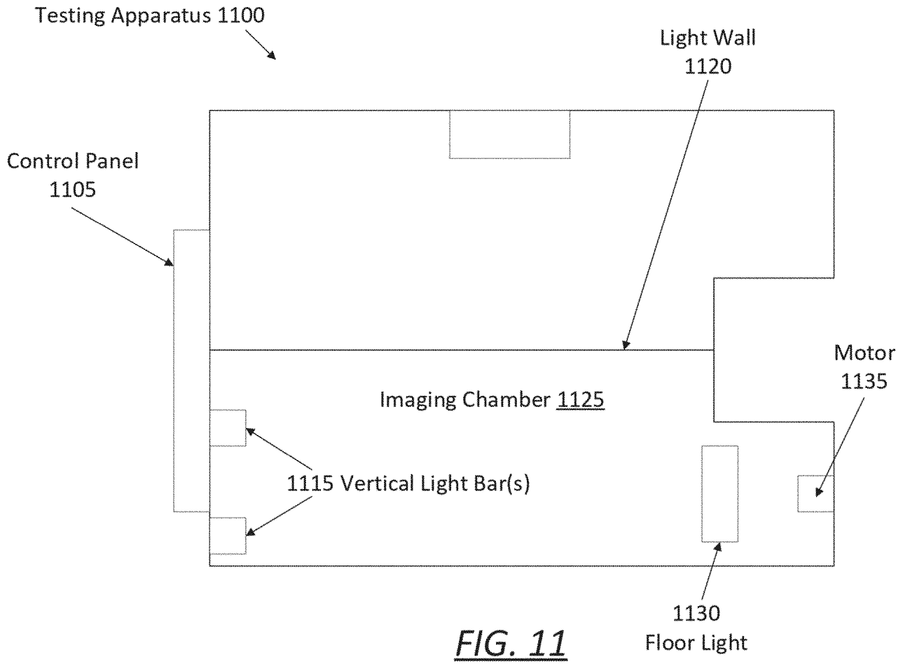

Turning to FIG. 11, an example of a top view of a testing apparatus (1100) is shown in accordance with one or more embodiments. As discussed above, the testing apparatus may include of a set of cameras and a multitude of light sources placed in different locations. As shown in FIG. 11, the testing apparatus (1100) includes of a control panel (1105) located on the outer portion of the testing apparatus. A light wall (1120) divides the testing apparatus (1100) into two separate chambers, a imaging chamber (1125) and a power chamber (not shown). The imaging chamber (1125) includes of vertical light bars (1115) located on the inside of a wall containing the control panel (1105), a floor light (1130) located opposite of the vertical light bars (1115), and a motor (1135).

In one or more embodiments, the vertical light bars (1115) are located on either side of a primary camera (not shown). For example, a vertical light bar (1115) may be on the right side of the camera and a second vertical light bar (1115) may be on the left side of the camera, opposite the first vertical light bar. The two vertical light bars (1115) may be LED lights or any other type of light that extend from the bottom of a wall to the top of the wall. In one or more embodiments, the vertical light bars (1115) are the same height of the camera. In one or more embodiments, the vertical light bars (1115) run along the entire height of the wall. The size of the vertical light bars (1115) should not be limited to these examples, and the size may be determined based on dimensions of the testing apparatus (1100) and the amount of light required to illuminate the imaging chamber (1125). In one or more embodiments, the vertical light bars (1115) may be connected to the camera via a wire, cable, or any other type of connection device. For example, the vertical light bars (1115) may receive an input from the camera via the wire to illuminate the imaging chamber (1125) when the camera captures in image. In one or more embodiments, the vertical light bars (1115) may be connected to an outside source, such as the control panel (1105). For example, the vertical lights (1115) may be programmed by the control panel (1105) to illuminate the hydrocarbon chamber when the camera captures an image.

In one or more embodiments, the floor light (1130) is located beneath the hydrocarbon test sample on the floor of the hydrocarbon test apparatus (1100). The floor light (1130) may be an LED strip or any other type of light source that illuminates the imaging chamber (1125). In one or more embodiments, the floor light (1130) may extend from one wall to an opposite wall. In one or more embodiments, the floor light (1130) may extend the length of the hydrocarbon test sample. The size of the floor light (1130) should not be limited to these examples, and the size may be determined based on dimensions of the testing apparatus (1100) and the amount of light required to illuminate the imaging chamber (1125). In one or more embodiments, the floor light (1130) may be connected to the camera via a wire, cable, or any other type of connection device. For example, the floor light (1130) may receive an input from the camera via the wire to illuminate the imaging chamber (1125) when the camera captures in image. In one or more embodiments, the floor light (1130) may be connected to an outside source, such as the control panel (1105). For example, the floor light (1130) may be programmed by the control panel (1105) to illuminate the hydrocarbon chamber when the camera captures an image.

In one or more embodiments, a motor (1135) is located on a wall adjacent to the hydrocarbon tests sample. The motor (1135) may be battery operated or connected to the power source. The motor (1135) is connected to the hydrocarbon test sample in a manner that will allow the motor to rotate the hydrocarbon test sample. For example, the motor (1135) may contain a belt which is joined between a rotating shaft on the motor and the hydrocarbon test sample. The rotation of the motor shaft will allow the turn the belt which will allow the hydrocarbon test sample to rotate.

In one or more embodiments, the motor (1135) may be located on a wall adjacent to the camera (not shown). The motor (1135) is connected to the camera in a manner that will allow the motor to rotate the camera. For example, the motor (1135) may contain a belt which is joined between a rotating shaft on the motor and the camera. The rotation of the motor shaft will allow the turn the belt which will allow the camera to rotate.

In one or more embodiments, the motor (1135) may be located on a wall adjacent to the vertical light bar (1115). The motor (1135) is connected to the vertical light bar (1115) in a manner that will allow the motor to rotate the vertical light bar (1115). For example, the motor (1135) may contain a belt which is joined between a rotating shaft on the motor and the vertical light bar (1115). The rotation of the motor shaft will allow the turn the belt which will allow the vertical light bar (1115) to rotate.

In one or more embodiments, the testing apparatus (1100) is divided in two separate chambers by a light wall (1120). The light wall (1120) runs the length of the testing apparatus (1100) from one wall to the opposite wall. For example, the light wall (1120) may be placed on the center point of the wall which contains the vertical light bars (1115) and run perpendicular from the wall, across the imaging chamber (1125), to the opposite wall. In one or more embodiments, the placement of the light wall (1120) is dependent upon the light conditions. Prior tests may be done to determine the placement of the light wall (1120) to provide the best illumination results for the imaging chamber (1125).

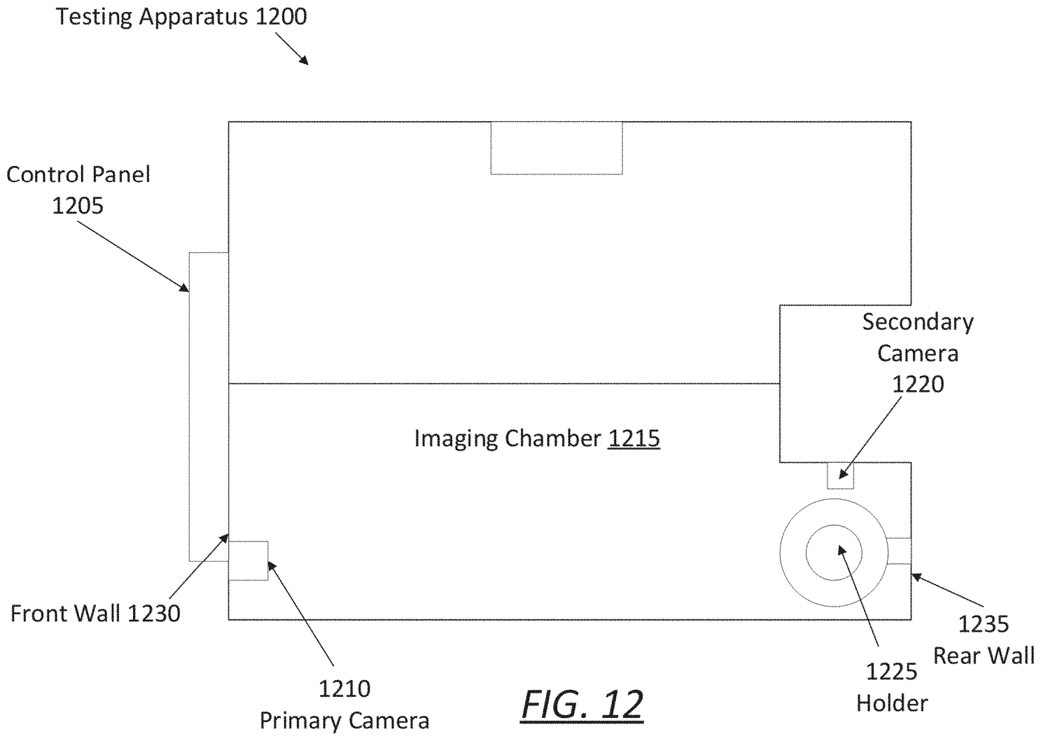

FIG. 12 shows an example of a top view of a testing apparatus (1200) in accordance with one or more embodiments. As discussed above, the testing apparatus (1200) may include of a set of cameras and a multitude of light sources placed in different locations. As shown in FIG. 12, the testing apparatus (1200) includes of a control panel (1205) and a imaging chamber (1215), which includes of a primary camera (1210) located on a front wall (1230) opposite of a hydrocarbon test sample (1240), which is positioned on a holder (1225), located on a rear wall (1235). A secondary camera (1220) is located on a wall perpendicular to the primary camera.

In one or more embodiments, the primary camera (1210) and the secondary camera (1220) capture a multitude of images of the hydrocarbon test sample (1240) and send the multitude of images to the control panel (1205) for analyzing. In one or more embodiments, the primary camera (1210) and secondary camera (1220) capture a multitude of images in unison. For example, the primary camera (1210) and secondary camera (1220) capture a multitude of images under the same lighting conditions at the same time. This allows for the multitude of images of the hydrocarbon test sample (1240) to be captured from multiple angles to provide greater accuracy in the analysis.

In one or more embodiments, the primary camera (1210) and secondary camera (1220) capture a multitude of images under different lighting conditions. For example, the primary camera (1210) may capture a multitude of images while the hydrocarbon test sample (1240) is illuminated by a UV light source and a multitude of other light sources. The secondary camera (1220) may then capture a multitude of images while the hydrocarbon test sample (1240) is illuminated by only the multitude of other light sources.

In one or more embodiments, the multitude of images captured by the primary camera (1210) and secondary camera (1220) are time stamped and tagged with a geographic location. For example, the primary camera (1210) and secondary camera (1220) may upload the multitude of images instantaneously to the control panel (1205) and the control panel assigns a time stamp and geographic location to the multitude of images using internal GPS. In one or more embodiments, the primary camera (1210) and secondary camera (1220) assign the time stamp and geographic location to the multitude of images before the multitude of images are uploaded to the control panel (1205).

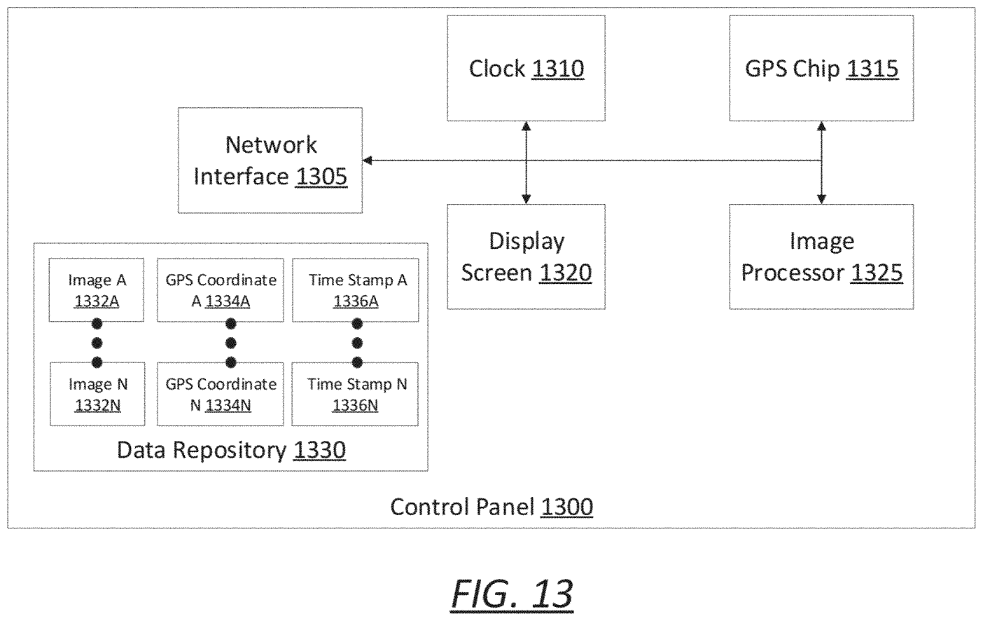

FIG. 13 shows a system in accordance with one or more embodiments. In one or more embodiments, the system is contained within a control panel (1300) which contains a network interface (1305), a data repository (1330), a display screen (1320), an image processor (1325), a clock (1310), and a Global Positioning System (GPS) chip (1315). Each of these components is described below.

As previously discussed, the control panel (1300) may be any type of input device (e.g., a computing tablet, a laptop, a mobile device, a computer). The control panel (1300) contains a data repository (1330) for storage. In one or more embodiments, the data repository (1330) is any type of storage unit and/or device (e.g., a file system, database, collection of tables, or any other storage mechanism) for storing data/information. Specifically, the data repository (1330) may include hardware and/or software. Further, the data repository (1330) may include multiple different storage units and/or devices. The multiple different storage units and/or devices may or may not be of the same type or located at the same physical site. Further, the data repository (1330) includes functionality to store, at least, multiple images (e.g., image A (1332A), image N (1332N)).

In one or more embodiments, multiple images (e.g., image A (1332A), image N (1332N)) are stored in the data repository (1330). Image A (1332A) may be an image captured of the hydrocarbon test sample using three light sources, while image N (1332N) may be an image of the hydrocarbon test sample captured using one light source and a UV light source.

In one or more embodiments, the data repository (1330) stores GPS coordinates (e.g., GPS coordinate A (1334A), GPS coordinate N (1334N)). The GPS coordinates may contain information related to the location of an image when the image is captured. The GPS coordinates may be assigned to an image when the image is captured. For example, image A (1332A) may be captured in a location and GPS coordinate A (1334A) contains the data associated with the location. GPS coordinate A (1334A) is then assigned to image A (1332A). Image N (1332N) may be captured in a second location and GPS coordinate N (1334N) contains the data associated with the second location. GPS coordinate N (1334N) is then assigned to image N (1332N).

In one or more embodiments, the data repository (1330) stores time stamps (e.g., time stamp A (1336A), time stamp N (1636N). The time stamps contain data related to the time that an image was captured. For example, image A (1332A) may be captured at 6:36 pm. Time stamp A (1336A) is then associated with 6:36 pm and image A (1332A). Image N (1332N) may be captured at 6:38 pm. Time stamp N (1636N) is then associated with 6:38 pm and image N (1332N).

Returning to the control panel (1300), the control panel contains a network interface (1305) to send and receive data. The network interface (1305) may be a local area network (LAN), a wide area network (WAN) such as the Internet, mobile network, Bluetooth, or any other type of network. The network interface (1305) may have capability to send and receive images to and from the control panel (1300) to be stored on a data repository (1330). For example, image A (1332A) may be captured by a camera and received by the control panel (1300) through Bluetooth using the network interface (1305). In one or more embodiments, the control panel (1300) may try to send image A (1332A) to a separate computing device without the network interface (1305) detecting a useable network. Image A (1332A) will remain in the data repository (1330) until the network interface (1305) detects a network to send image A (1332A) to the separate computing device.

The control panel (1300) may contain an image processor (1325). The image processor (1325) is configured to perform instructions on the control panel (1300) (e.g., image processing, algorithms). The image processor(s) (1325) may be an integrated circuit for processing instructions. For example, the image processor(s) may be one or more cores, or micro-cores of a processor.

The control panel (1300) may contain a display screen (1320) to display content to the user. The display screen may be a liquid crystal display (LCD), a plasma display, touchscreen, cathode ray tube (CRT) monitor, projector, or other display device. For example, the display screen (1320) may display image A (1332A) to the user. In one or more embodiments, the user takes actions based on being presented image A (1332A). The user may choose to send image A (1332A) to a separate computing device via the network face (1305), choose to store image A (1332A) in the data repository (1330), or choose to run image processing based on the instructions in the image processor (1325).

In one or more embodiments, the control panel (1300) may contain a clock (1310). The clock (1310) may display the time to the user and track time internally on the control panel (1300). For example, the clock (1310) may provide time stamp A (1336A) to image A (1332A) based on the time that image A was captured by the control panel (1300).

In one or more embodiments, the control panel (1300) may contain a GPS chip (1315). The GPS chip (1315) has capability to track the location of the control panel (1300) and to store the information in the data repository (1330). For example, the GPS chip (1315) may provide the location of image A (1332A) in the form of GPS coordinate A (1334A) based on the location that image A (1332A) was captured by the control panel (1300).

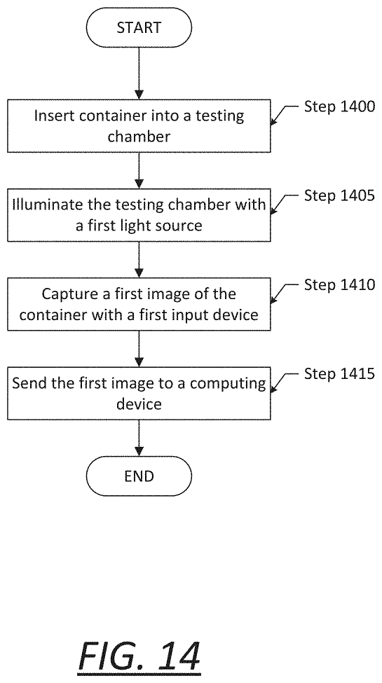

Turning to FIG. 14, a flowchart describing a method for capturing an image of a container is shown. In step, 1400, a container is inserted into a test chamber. The testing chamber may contain a lid that opens to allow the container to be inserted. In one or more embodiments, the testing chamber may rotate to reveal an opening for the container to be inserted.

In step 1405, the testing chamber is illuminated by a first light source. In one or more embodiments, the first light source is directed towards the container in the testing chamber. In one or more embodiments, the first light source is directed towards a reflective wall that allows for the light to reflect off the wall onto the container. In one or more embodiments, one or more light sources may be used to illuminate the testing chamber.

In step 1410, a first image of the container is captured by a first input device. The first input device is located in a manner that allows for the image contents to contain the entire container. In one or more embodiments, a second input device captures an image of the container. In one or more embodiments, multiple images of the container are captured.

In step 1415, the first image is sent to a computing device from the first input device. The first image is initially stored on the first input device. The first image is then sent to the computing device over a network (e.g., Bluetooth, a LAN connection network, WiFi). In one or more embodiments, the computing device is a control panel connected to the testing apparatus. In one or more embodiments, the computing device is external from the testing apparatus.

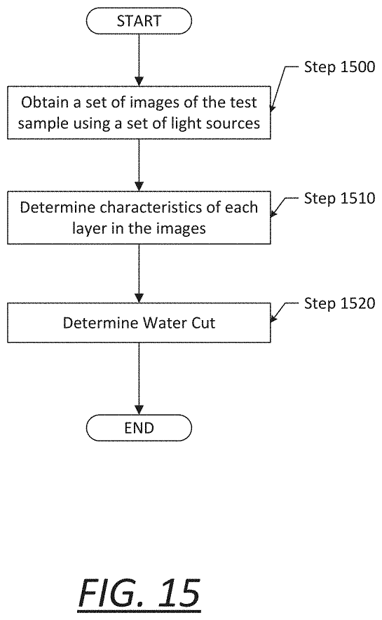

Turning to FIG. 15, a flowchart describing a method for determining a water cut is shown. In step 1500, a set of images is obtained by a set of cameras using a set of light sources. In one or embodiments, multiple images of the test sample are obtained by the set of cameras. For example, the set of cameras may take a first image and a second image of the test sample under different lighting conditions from the light sources.

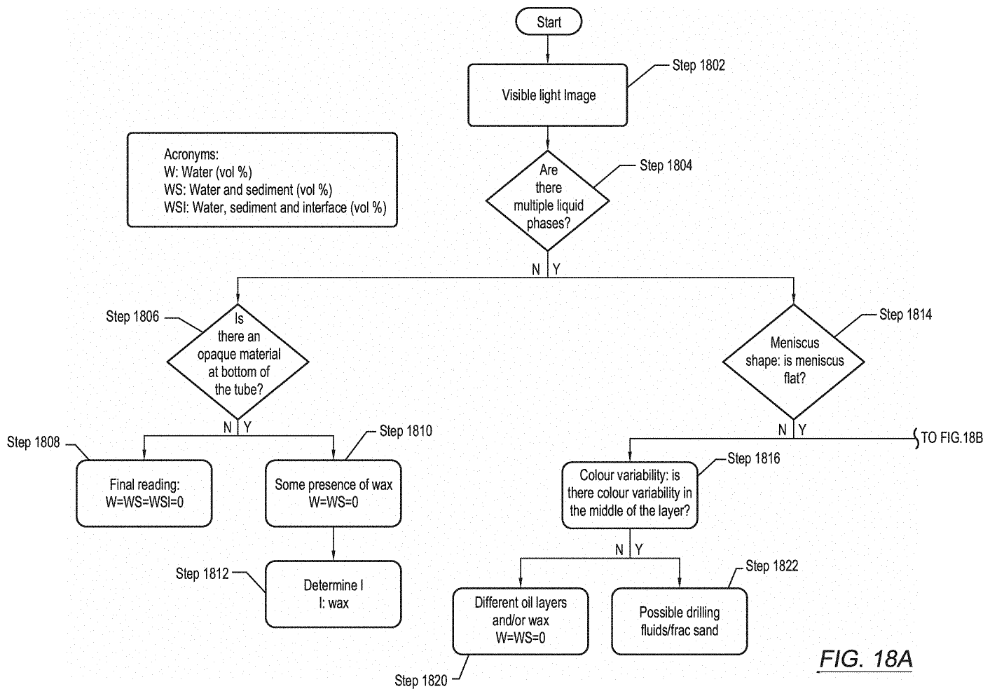

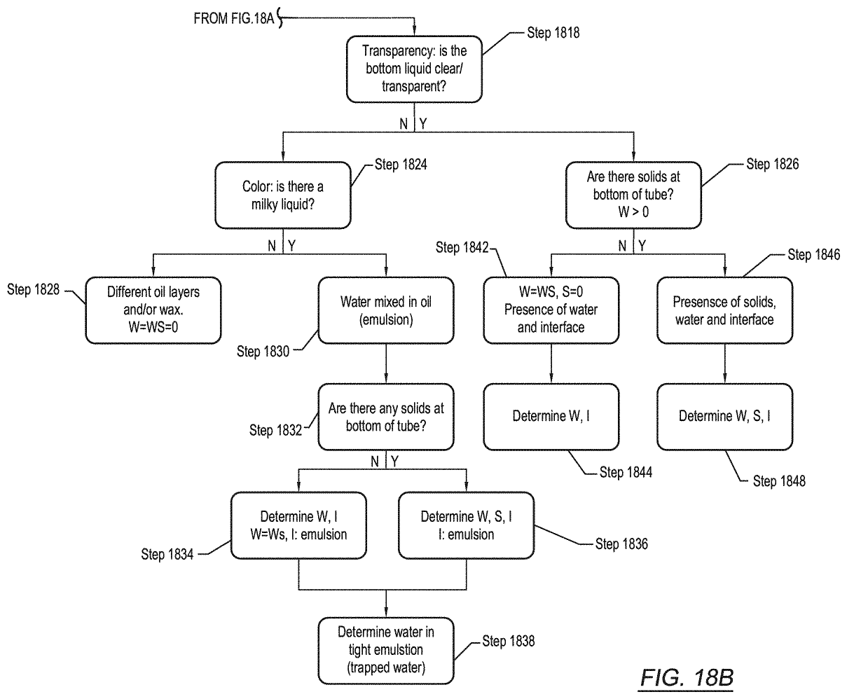

In step 1510, the characteristics of each layer of the multiple images is determined. In one or more embodiments, the set of cameras may send the multiple images directly to the control panel over a network (e.g., Wi-Fi, Bluetooth, cellular network). The control panel may use image processing to determine the characteristics of the layers for the multiple images. For example, the control panel may determine the boundary layers of an image and determine the properties of each boundary layer. The image processing and boundary layers are discussed in further detail below and in FIGS. 18A and 18B.

In step 1520, a water cut is determined based on the results from step 1510. The water cut is the percentage of water found in the test sample. In one or more embodiments, the control panel processes the multiple images to determine the water cut. In one or more embodiments, a user assists in determining the water cut and verifying the results from the control panel. The water cut is discussed in further detail below and in FIGS. 18A and 18B.

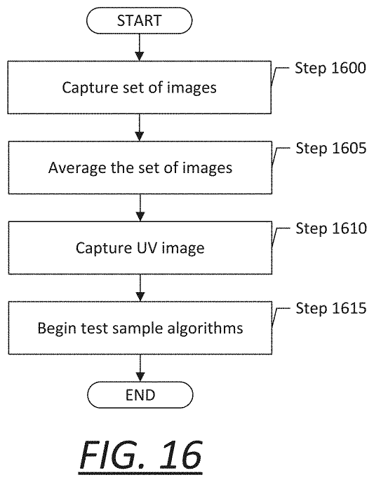

FIG. 16 shows a flowchart describing a method for capturing a set of images to feed into an algorithm. Step 1600 involves capturing a set of images of the test sample inside of the testing apparatus. Multiple images may be captured by a single camera or both a primary camera and a secondary camera. In one or more embodiments, different groups of one or more light sources are used to illuminate the testing apparatus to assist in capturing the multiple images.

In step 1605, the set of images are averaged to produce a visible light image. In one or more embodiments, the control panel obtains multiple images from the set of cameras. The control panel may use image processing to process the multiple images to form an average visible light image. For example, the control panel may determine the boundary layer for each image from the multiple images and determine the average boundary layer across the multiple images. An average visible light image is formed by the control panel using the average boundary layers.

In step 1610, a UV image is captured by the set of cameras. In one or more embodiments, the testing apparatus contains a UV light source. The UV light source may illuminate the test sample while the set of cameras capture an image. In one or more embodiments, the UV light source is the only light source illuminating the test sample.

In step 1615, the control panel has obtained the average visible light image and the UV image to begin the algorithms. In one or more embodiments, the control panel has the average visible light image and the UV image stored locally. In one or more embodiments, the control panel may send the average visible light image and the UV image to a separate computing device to process the algorithms. For example, the control panel may send the average visible light image and the UV image over a wireless network to a separate computing device located away from the control panel.

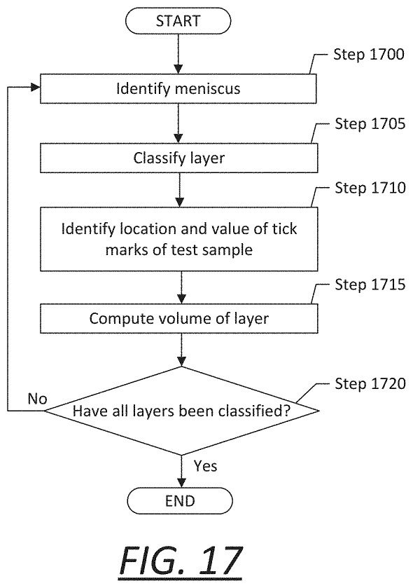

Turning to FIG. 17, a flowchart describing a method for analyzing layers of a sample by computing the volume layer of the test sample is shown. In step 1700, the meniscus of the test sample is identified. The meniscus may be flat or curved and may be identified by image processing or by the user examining the meniscus. More detail about identifying the meniscus may be found in FIGS. 18A and 18B.

In step 1705, the layers of the of the image are classified. In one or more embodiments, the layer is a liquid or solid phase that is present in the test sample. The liquid phase or solid phase may be water, oil, drilling fluid, sand, mud, sediment, was, emulsion, or any other type of solid or liquid that may be present in oil drilling. The solids and liquids are separated into layers using a centrifuge. In one or more embodiments, the layers are examined by the control panel using image processing. In one or more embodiments, the user examines the image to identify the layers of the test sample. More detail about classifying the layers of the test sample may be found in FIGS. 18A and 18B.

In step 1710, the location of volume marks on the test sample are identified. In one or more embodiments, the test sample is a container filled with the sample. The container may be a test tube, a cylindrical tube, a flask, a beaker, or any other type of container that holds a liquid. For example, the container may be a tube that is manufactured in accordance with the ASTM D4007 or the ASTM D0097 standards published by ASTM International. The container may be made of glass, plastic, acrylic glass, or any other type of material which allows for the user or control panel to see the liquid and solid phases. In one or more embodiments, the container is labeled with volume marks to represent the volume of the container. The volume marks are located in a manner to be visible by the set of cameras. The volume may be indicated in liters, milliliters, cubic centimeters (cm³), fluid ounces, or any other type of measure for volume.

In one or more embodiments, the control panel recognizes the volume marks on the image via image processing. The location of the volume marks for the layer is recorded by the control panel to be used for calculating volume, as described in Step 1715. In one or more embodiments, the user identifies the volume marks and stores the values to be used for calculating the volume.

In Step 1715, the volume of the layer is calculated. In one or more embodiments, the control panel calculates the volume of the layer based on the volume mark values recorded in the previous step. The dimensions of the container of the test sample are known by the control panel, so the correct formula to calculate volume of the container is applied. In one or more embodiments, the user calculates the volume.

In Step 1720, a determination is made if all the layers have been classified. Each layer of the test sample is identified, and the volume is calculated for each layer before the process may end. If not all the layers have been classified, the process starts again at step 1700 for the next layer. If all layers have been classified and the respective volumes have been calculated, the process ends.

FIGS. 18A and 18B show a flowchart describing a method for determining water volume percentage in a test sample. In Step 1802, an image is captured using the camera while being illuminated by the light sources. The image is then sent to the control panel. In one or more embodiments, the image is sent to the control panel over a network (e.g., Wifi, cellular network, bluetooth). In one or more embodiments, the camera is connected to the control panel via a connection cable (e.g., USB cable, ethernet cable, VGA cable). In one or more embodiments, the multitude of images may be uploaded from the camera onto a portable storage device (e.g., USB drive, portable hard drive). The multitude of images are then transferred to the control panel from the portable storage device by inserting the portable storage device into the control panel. In one or more embodiments, the camera is the control panel. In this case, the multitude of images would be taken by the control panel and locally stored on the control panel with no need for other input.

In Step 1804, a determination is made as to whether there are multiple liquid phases detected in the image. The image may be processed by the control panel to analyze the number of liquid phases in the test sample. For example, the control panel may determine that there is only one liquid phase in the test sample. In this case, the process moves to Step 1806. If the control panel determines there are multiple liquid phases from the image, the process moves to Step 1814.

In one or more embodiments, the image is sent from the control panel to an external computer device, such as a laptop, computer, tablet computing device, or mobile phone. The image may be sent over a network, as described above, a connection cable, or a portable storage device. For example, the external computer device may be located at another location on the site, so the image is sent from the control panel over the a secure WiFi network to the external computer. In one or more embodiments, the control panel may be located outside while the external computer device is located inside. In one or more embodiments, the image is viewed on the external computer device by a user instead of or in addition to being analyzed by the external computer device. The user determines if multiple liquid phases are present in the image and sends feedback to the external computer device via an input device such as a keyboard, mouse, voice recognition, or any other type of device that inputs data into the external computer device. The image may then be sent back to the control panel over a network to continue the process. In one or more embodiments, the process continues without sending the image back to the control panel since the information sent to the external computer device is sufficient to carry on the process.

In one or more embodiments, the image may remain on the control panel but is not processed by the control panel. For example, the image may be displayed on the control panel and a user views the image to determine if multiple layers are present.

Turning to Step 1806, the analysis of the image determines that multiple liquid phases are not present in Step 1804, and a determination is then made as to whether there is an opaque material at the bottom of the test sample. In one or more embodiments, the control panel determines if the bottom of the test sample is opaque through image processing. If the control panel determines that the bottom of the test sample is opaque, wax is present in the test sample and the process ends at Step 1808. If the control panel determines that there is no opaque material in the bottom of the test sample, the water volume percentage is set to 0 at Step 1810 and the process ends by determining the interface volume percentage at Step 1812.

In one or more embodiments, the analysis may be done by a user at the control panel. The user views the image on the control panel to determine if there is an opaque material present in the test sample. In one or more embodiments, the analysis is done by a user at an external computer device.

In Step 1814, the analysis determines that multiple liquid phases are present in the image in Step 1804, and a determination is made if the meniscus shape is flat in the test sample. The control panel analyzes the image via image processing to make a determination of the shape of the meniscus. When the meniscus is not flat, the process continues to Step 1816. When the meniscus is flat, the process continues to Step 1818.

In one or more embodiments, the analysis is carried out by a user at the control panel. The user views the image on the control panel and makes a determination in regard to the shape of the meniscus. In one or more embodiments, the analysis is carried out by a user at an external computer device.

In Step 1816, the analysis determines that the meniscus shape was not flat in Step 1814, and a determination is made if there is color variability in the middle of the layer of the test sample. The control panel analyzes the image via image processing to make a determination if there is color variability in the middle layer of the image. When there is no color variability, the analysis concludes that there are different oil layers and/or wax present in the test sample and the process ends at Step 1820 setting the water and water volume percentage and the water and sediment volume percentage to 0. When there is color variability in the middle layer, the analysis concludes that there is possible drilling fluid or frac sand in the test sample and the process ends at Step 1822.

In one or more embodiments, the analysis is carried out by a user at the control panel. The user views the image on the control panel and makes a determination in regard to color variability of the middle layer of the test sample. In one or more embodiments, the analysis is carried out by a user at an external computer device.

Turning to Step 1818, the analysis determines that the meniscus is flat in Step 1814, and a determination is then made as to whether the liquid on the bottom of the test sample is clear or transparent. The control panel analyzes the image via image processing to make a determination if there is clear or transparent liquid on the bottom of the test sample. When there is no clear liquid, the process moves to Step 1824 for further analysis. When there is clear liquid, the process moves to Step 1826 for further analysis.

In one or more embodiments, the analysis is carried out by a user at the control panel. The user views the image on the control panel and makes a determination in regard to the transparency of the liquid in the bottom of the test sample. In one or more embodiments, the analysis is carried out by a user at an external computer device.

In Step 1824, the analysis determines that the liquid is not clear on the bottom of the test sample in Step 1818, and a determine is made if the liquid is milky. The control panel analyzes the image via image processing to make a determination if the liquid is milky. When the analysis reveals that the liquid is not milky, a determination is made that there are potentially different oil layers and/or wax in the test sample and the process ends at Step 1828 setting the water and water volume percentage and the water and sediment volume percentage to 0. When the analysis reveals that the liquid is milky, then a determination is made at Step 1830 that there is water mixed in the oil and potential for emulsion, and the process continues to Step 1832.

In one or more embodiments, the analysis is carried out by a user at the control panel. The user views the image on the control panel and makes a determination in regard to the liquid being milky in the test sample. In one or more embodiments, the analysis is carried out by a user at an external computer device.

In Step 1826, the analysis determines that the liquid is clear on the bottom of the test sample in Step 1818, and a determination is made if there are solids at the bottom of the tube. The control panel analyzes the image of the test sample via image processing to determine if there are solids present in the bottom of the test sample. When the analysis reveals that solids are not present, then the process proceeds to Step 1842. When the analysis reveals that solids are present, then the process proceeds to Step 1846.

In Step 1832, the image of the test sample is analyzed via image processing to determine if there are solids present in the bottom of the test sample. When the analysis reveals that solids are not present, then at Step 1834 the water content and interface level are calculated at Step 1838 based on the analysis from the image processing. When the analysis reveals that solids are present in the test sample, then at Step 1836 the water content, solid content, and interface level are all calculated at Step 1838 based on the analysis from the image processing.

In one or more embodiments, the analysis is carried out by a user at the control panel. The user views the image on the control panel and makes a determination in regard to the amount of solids at the bottom of the test sample. In one or more embodiments, the analysis is carried out by a user at an external computer device.

In Step 1842, the water volume percentage is determined to be equal to the water and the sediment volume percentage is determined to be 0. The water content and interface level are then calculated based on the analysis from the image processing at Step 1844.

In Step 1846, it is determined that solids, water, and interface are present. The water content, solid content, and interface level are then all calculated based on the analysis from the image processing at Step 1848.

In one or more embodiments, the analysis is carried out by a user at the control panel. The user views the image on the control panel and makes a determination in regard to the amount of solids at the bottom of the test sample. In one or more embodiments, the analysis is carried out by a user at an external computer device.

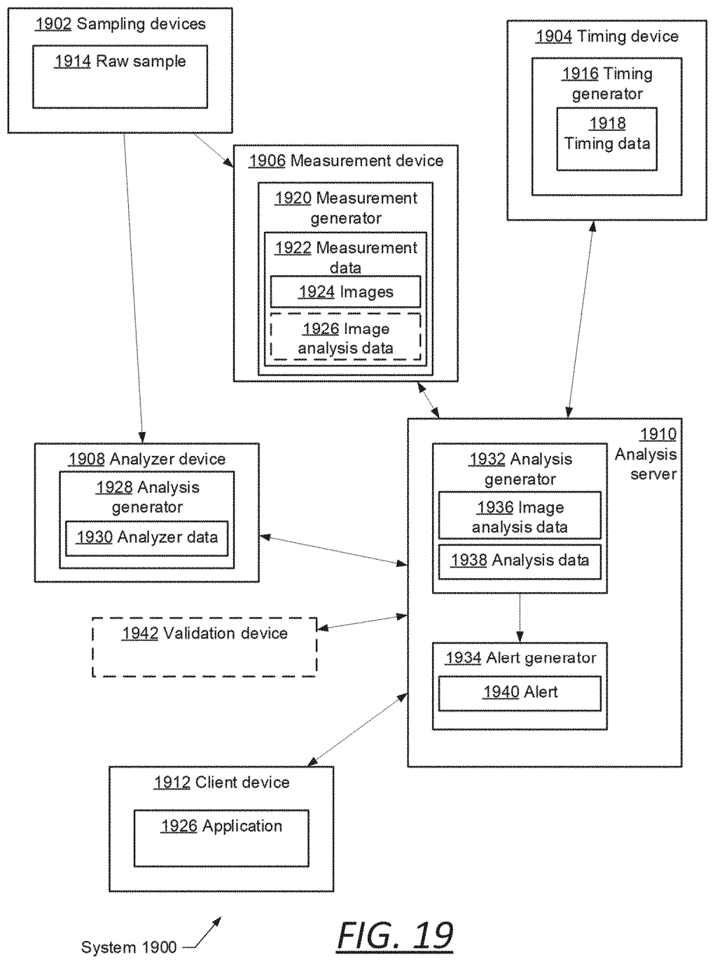

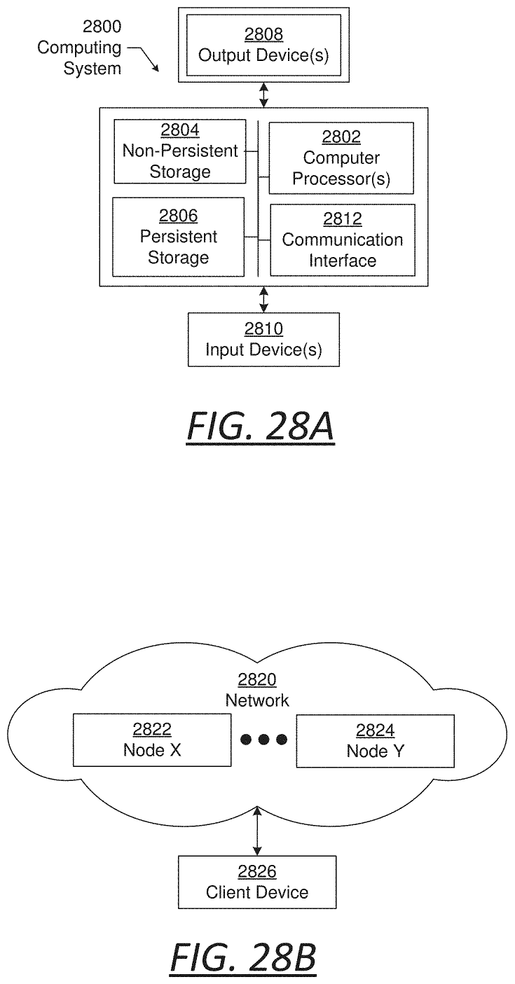

FIG. 19 shows a diagram of a computing system (1900) in accordance with one or more embodiments of the invention. The computing system (1900) may correspond to the computing system shown in FIGS. 28A and 28B. In particular, the type, hardware, and computer readable medium for the computing system (1900) is presented in reference to FIGS. 28A and 28B. FIG. 19 shows a component diagram of the computing system (1900). The computing system (1900) includes a set of component devices, including sampling devices (1902), timing device (1904), measurement device (1906), analyzer device (1908), validation device (1942), client device (1912), and analysis server (1910).

The sampling device (1902) collects raw samples (1914) that are processed and analyzed with one or more of the measurement device (1906) and the analyzer device (1908). In one or more embodiments, the sampling device (1902) is a graduated cylinder and the raw sample (1914) is a hydrocarbon test sample.

The timing device (1904) is communicatively connected to the analysis server (1910). In one or more embodiments, the timing device (1904) is a portable device, such as a tablet computer, that is carried to each location of a sampling event. The timing device (1904) is accessed each time a raw sample (1914) is taken with a sampling device (1902) and at each point and location of the processing and handling of the raw sample (1914) to generate the timing data (1918) with optional location data. The timing device (1904) includes the timing generator (1916). The timing generator (1916) includes one or more hardware and software modules to generate and provide the timing data (1920), such as a real time clock and a global positioning system (GPS) receiver. The timing data (1918) includes a date and time for each sampling event of a set of sampling events. In additional embodiments, the timing data (1918) also includes location data, such as GPS coordinates of the timing device (1904), to record the time, date, and location of the timing device (1904) when the timing device (1904) is accessed to log a sampling event.

The measurement device (1906) is a testing apparatus that is connected to the analysis server (1910). In one or more embodiments, the measurement device (1906) is a centrifugal tube reader that is in accordance with the testing apparatus described above in FIGS. 1 through 12. The measurement device (1906) includes a measurement generator (1920) that generates measurement data (1922) based upon the raw sample (1914). In one or more embodiments, the measurement data (1922) includes a set of images (1924) of the raw sample (1914) and optionally includes image analysis data (1926). In one or more embodiments, the set of images (1924) includes a set of raw images that were captured with a camera of the measurement device (1906) and includes one or more processed images that are generated by processing the set of raw images, as described in the methods of FIGS. 15, 16, 17, 18A and 18B. The image analysis data (1926) includes a water percentage value, a settlement percentage value, and a water/oil interface percentage value.

The analyzer device (1908) is connected to the analysis server (1910) and includes the analysis generator (1928). The analysis generator (1928) includes one or more hardware and software modules that operate to generate the analyzer data (1930). In one or more embodiments, the analyzer data (1930) includes a set of values for temperature (T(t)), density (ρ(t)), water characteristics (Waterₚₕₐₛₑ(t), Water_{DRX}(t), Water_{NOC}(t)), flowrate (Flowrate(t)), and volume (Volume(t)).

The analysis server (1910) is connected to a set of devices (1904, 1906, 1908, 1942, 1912) using one or more network connections. In one or more embodiments, the analysis server (1910) includes an analysis generator (1932) and an alert generator (1934). The analysis generator (1932) generates the image analysis data (1936) and the analysis data (1938). The analysis generator may also process the set of images (1924) to generate the processed image, which is used to generate the image analysis data (1936). The image analysis data (1936) may be a copy of the image analysis data (1926) from the measurement device (1906). The alert generator (1934) generates an alert (1940) based on the analysis data (1938). The analysis data (1938) includes one or more process recommendations, error probabilities, and failure mode analysis.

The client device (1912) is connected to the analysis server (1910) with a network connection. The client device (1912) includes an application (1926) that allows for interaction with the system (1900).

The validation device (1942) is connected to the analysis server (1910). The validation device is used to validate the image analysis data (1936) and the analysis data (1938).

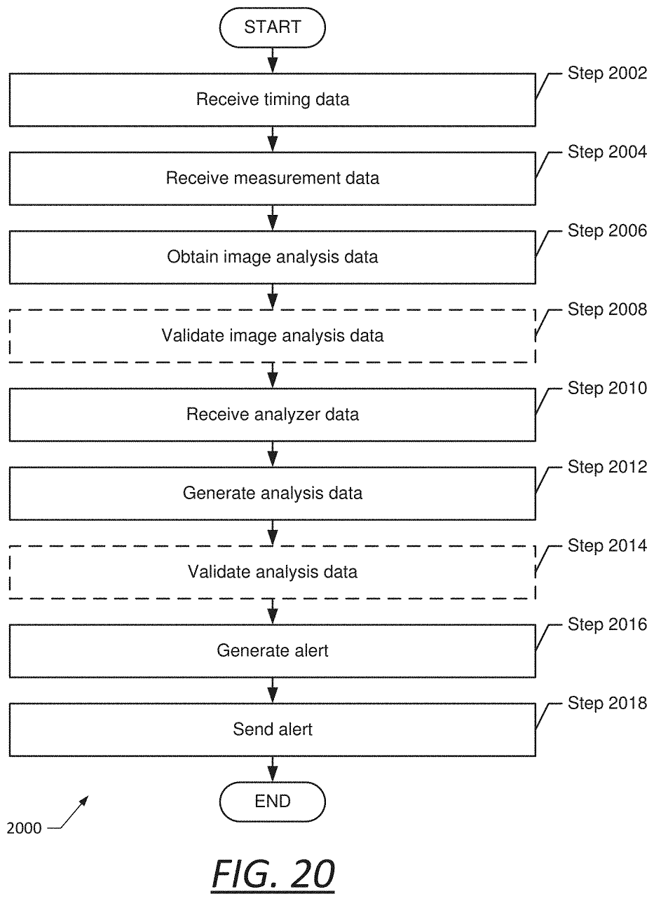

FIG. 20 shows a flowchart describing a method (2000) for analyzing samples. In one or more embodiments, steps of the method (2000) are performed by the analysis server (1910) of the system (1900) of FIG. 19.

In Step 2002, timing data is received. In one or more embodiments, the timing data (1918) is received by the analysis server (1910) and provided by the timing device (1904). The timing device (1904) generates the timing data (1918) with the timing generator (1916). The timing device (1904) is accessed each time a raw sample (1914) is taken with a sampling device (1902) and at each point of the processing and handling of the raw sample (1914). In one or more embodiments, the timing device (1904) is accessed by a user interacting with an application on the timing device (1904) to select and identify a type of sample being taken and the action being performed.

The timing generator (1916) of the timing device (1904) logs each access and records the time, date, and action for each step. For example, the timing generator (1916) generates log entries for when the raw sample (1914) is originally taken from a sampling device (1902), when the raw sample (1914) is placed into the measurement device (1906), and when the raw sample (1914) is removed from the measurement device (1916). In one or more embodiments, the timing device (1904) also records the location of the timing device (1904) for each access.

In Step 2004, measurement data is received. In one or more embodiments, the measurement data (1922) is received by the analysis server (1910) from the measurement device (1906). The measurement data (1922) is generated by the measurement generator (1920). The measurement generator (1920) generates a set of images (1924) of the raw sample (1914) and optionally processes the set of images to generate image analysis data (1926). After generating the measurement data (1922) by capturing the set of images (1924) and optionally generating the image analysis data (1926), the measurement device (1906) sends the measurement data (1922) to the analysis server (1910).

In Step 2006, image analysis data is obtained. In one or more embodiments, the image analysis data (1938) is obtained by either receiving the image analysis data (1926) from the measurement device (1906) or by generating the image analysis data (1936) with the analysis generator (1932). Generation of the image analysis data is discussed further in the methods of FIGS. 15, 16, 17, 18A and 18B.

In optional Step 2008, image analysis data is validated. In one or more embodiments, the image analysis data (1926, 1938) is validated by a human operator of the measurement device (1906) or the validation device (1942). To validate the image analysis data, a processed image generated from the set of images (1924) is displayed with the image analysis data (1936). A selection is then received that identifies the validity of the image analysis data (1936). When the selection indicates that the image analysis data (1936) is not valid, the process ends.

In Step 2010, analyzer data is received. In one or more embodiments, the analyzer data (1930) is received by the analysis server after being sent by the analyzer device (1908). The analyzer data (1930) is generated with the analyzer device (1908) by processing the data related to the raw sample (1914) with the analysis generator (1928).

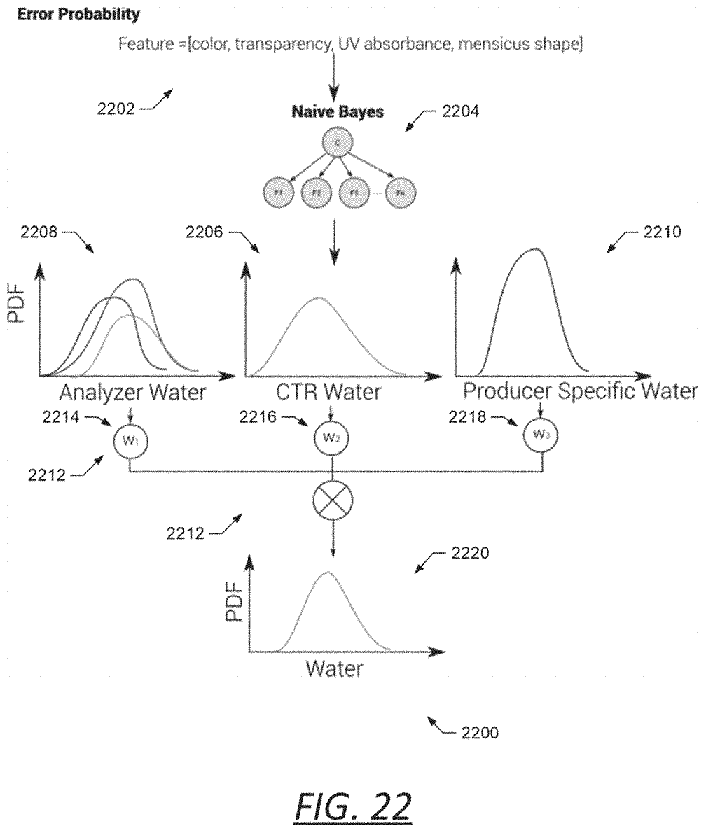

In Step 2012, analysis data is generated. In one or more embodiments, the analysis data (1938) is generated by the analysis generator (1932) by processing the timing data (1918), the measurement data (1920), the analyzer data (1930), and the image analysis data (1936), which is further described in the methods of FIGS. 22, 23, 24, 25, 26, and 27.

In optional Step 2014, analysis data is validated. In one or more embodiments, the analysis data (1938) is validated by a human operator of the validation device (1942). The analysis data (1938) is transmitted to and displayed by the validation device (1942). A set of selections are received by the validation device (1942) that indicate the validity of the analysis data (1938). When a selection indicates that the analysis data (1938) is not valid, the process ends.

In Step 2016, an alert is generated. In one or more embodiments, the alert (1940) is one of a set of alerts created by the alert generator (1934) of the analysis server (1910). The alerts are created in response to the analysis data (1938) based on a set of rules. In one or more embodiments, the rules specify: a set of process recommendations that when provided will trigger an alert, a range of error probabilities that when exceeded trigger an alert, and a set of failure mode analyses that when provided will trigger an alert.

In Step 2018, an alert is sent. In one or more embodiments, the alert (1940) is sent from the analysis server (1910) to the client device (1912) and is displayed by the application (1926).

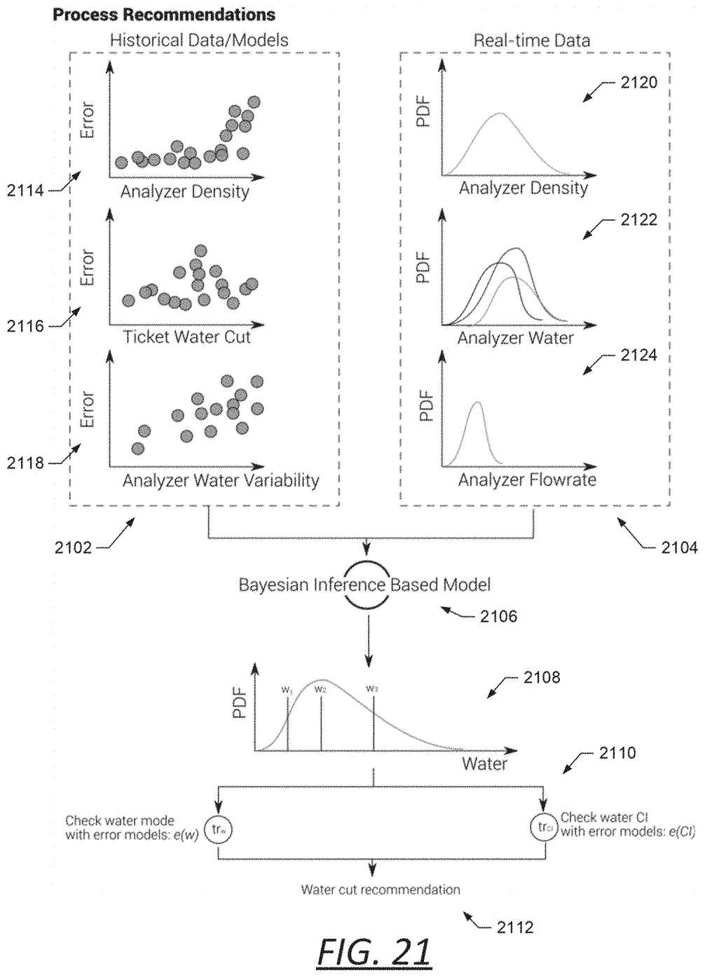

FIG. 21 shows a diagram of a method for generating process recommendations. In one or more embodiments, the method (2100) is performed by the analysis generator (1932) on the analysis server (1910) to generate a set of process recommendations in the analysis data (1938). In one or more embodiments, the diagram is displayed by the application (1926) of the client device (1912).

A set of historical data (2102) and a set of real-time data (2104) are processed using a Bayesian inference based model (2106) to generate a probability distribution function (2108). The probability distribution function (2108) is checked against a set of error models (2110) from which a set of recommendations (2112) are generated.

In one or more embodiments, the set of historical data (2102) includes data that was provided by the analyzer device (1908) and data that was generated by processing the data from the analyzer device (1908). For example, the set of historical data (2102) can include a set of analyzer density error data (2114), a set of ticket water cut error data (2116), and a set of analyzer water variability error data (2118).

In one or more embodiments, the set of real-time data (2104) includes data that is provided by the analyzer device (1908) and was optionally processed by the analysis generator (1932). For example, the set of real-time data (2104) can include an analyzer density probability distribution function (2120), a set of analyzer water probability distribution functions (2122), and an analyzer flowrate probability distribution function (2124).

In one or more embodiments, the Bayesian inference based model (2106) combines the historical data (2102) and the real-time data (2104) by 1) collecting the data at a producer- and/or production site-level; 2) cleaning the data by removing potential outliers using one or more preset thresholds, interquartile-range considerations, and comparisons to previously generated distributions; and 3) using the data to train density-estimation machine learning techniques (such as kernel density estimation) and Bayesian techniques to produce prior probability distributions. The data may first be transformed via mathematical functions to fit specific Bayesian modelling techniques, such as Bayesian Linear Regression.

In one or more embodiments, the probability distribution function (2108) generated using the Bayesian inference based model (2106) indicates the percentage of water in a sample. The probability distribution function (2108) is checked against a set of error models (2110) by determining if the input values for the probability distribution function correspond to a predetermined threshold probability for error in the on-line analyzer values. For example, if the current input values correspond in the error model to a probability of error in measurement of 65%, and the predetermined threshold for this particular instrument is 60%, then the recommendation would be made to take a spot sample or spot series to determine the true value of the percentage of water. The error models are trained on previous data points that contain both analyzer measurements as well as centrifuge measurements, using methods such as Naive Bayes classification/regression, decision trees, and random forests, to model the discrepancy between analyzer value and true value based on input values (e.g., analyzer density measurement, etc.), and assign probabilities to the existence of errors. Furthermore, the probability densities for water values are used to determine recommendations based on ranges for values. For example, if a given site regularly receives water percentages ranging from 0 to 4%, and the current values suggested a high probability of water at 0.1%, with a 70% chance of error in the range of +/−0.15% water, a recommendation for sampling may not be as important as it would to a site who regularly receives water percentages ranging from 0 to 0.5%.

FIG. 22 shows a diagram of a method for generating error probabilities. In one or more embodiments, the method (2200) is performed by the analysis generator (1932) on the analysis server (1910) to generate an error probability as a probability distribution function as a part of the analysis data (1938). In one or more embodiments, the diagram is displayed by the application (1926) of the client device (1912).

A set of features (2202) is extracted. In one or more embodiments, the set of features (2202) are extracted by processing the set of images (1924) to generate the image analysis data (1936) and include color, apparently, UV absorbance, and meniscus shape.

The set of features (2202) are combined using a naive Bayes classifier to form the probability distribution function (2206) by training the classifier using labels associated to given groups of feature data to create probability distributions with Bayes' Theorem for each feature value to exist in each of the given possible classifying labels. This allows for combinations of features previously unseen by the model to have probabilities associated for membership in each of the classifying labels.

In one or more embodiments, the probability distribution function (2206) is fed into an analytical model (2212) with a set of analyzer water probability distribution functions (2208) generated with data provided by the analyzer device (1908) and a producer specific water probability distribution function (2210). The producer specific water probability distribution function (2210) was generated by comparing the real-time data and the historical distribution of real-time data and spot sample readings from that specific producer. The distribution of discrepancies between the spot sample readings and the historical real-time readings taken at or estimated to be from the time that the spot sample was taken determines the probability distribution of possible offsets between the true value and the measured real-time value.

The analytical model (2212) includes a set of weights (2114). Each weight (2114) is applied to one or more probability distribution functions that are fed into the analytical model (2212). The weighted probability distribution functions are then combined to generate an error probability formed as the probability distribution function (2220).

The weights of the analytical model (2212) are determined by utilizing optimization methods on the space of possible weight values. The optimization methods include, but are not limited to: grid-search, gradient descent, and differential evolution. The optimization methods are validated using techniques such as cross-validation and time-directed walk-forward analysis on existing data that is divided into training and test sets.

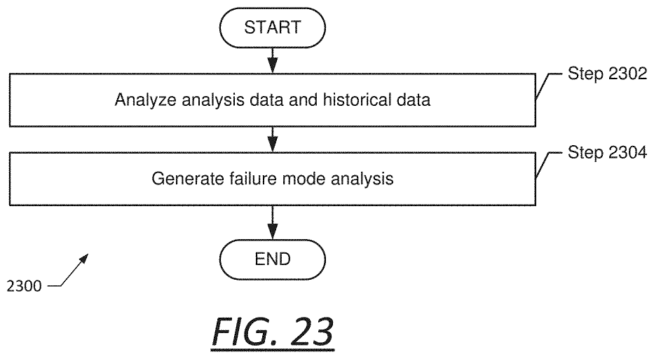

FIG. 23 shows a flowchart of a method for generating a failure mode analysis. In one or more embodiments, generation of the failure mode analysis is performed by the analysis generator (1932) of the analysis server (1910) as a part of or after generating the analysis data (1938).

In Step 2302, the analysis data and the historical data are analyzed. In one or more embodiments, the historical data includes all of the timing data (1918), images (1924), image analysis data (1926, 1936), analyzer data (1930), and analysis data (1938) that have been generated with the system (1900).

In one example, the failure mode analysis analyzes the timing data for inconsistencies. One inconsistency is when the time between when a sample is taken from an offload and when the sample is placed into the measurement device is too large. When the actual time taken between these two steps is above a predetermined threshold, then the measurement data (1922) provided by the measurement device (1906) may be inaccurate. The predetermined threshold may be derived from the historical data by calculating the average and standard deviation for the time between these two steps. The standard score of actual time taken is determined by subtracting the average from the actual time and then dividing the subtraction result by the standard deviation.

In Step 2302, the failure mode analysis is generated. In one or more embodiments, the failure mode analysis identifies how a failure occurred in the system (1900) for which a process recommendation can be provided.

From the example above, when the standard score is above a certain limit, e.g., 1, 2, or 3, the failure mode analysis identifies that the time between taking the sample and testing the sample is a likely cause for unreliable measurement data (1922) from the measurement device (1906). The analysis can be displayed on the application (1926) of the client device (1912).

In one or more embodiments, the failure mode analysis that is generated can include a set of figures. The set of figures can be transmitted to and displayed by the application (1926) of the client device (1912).

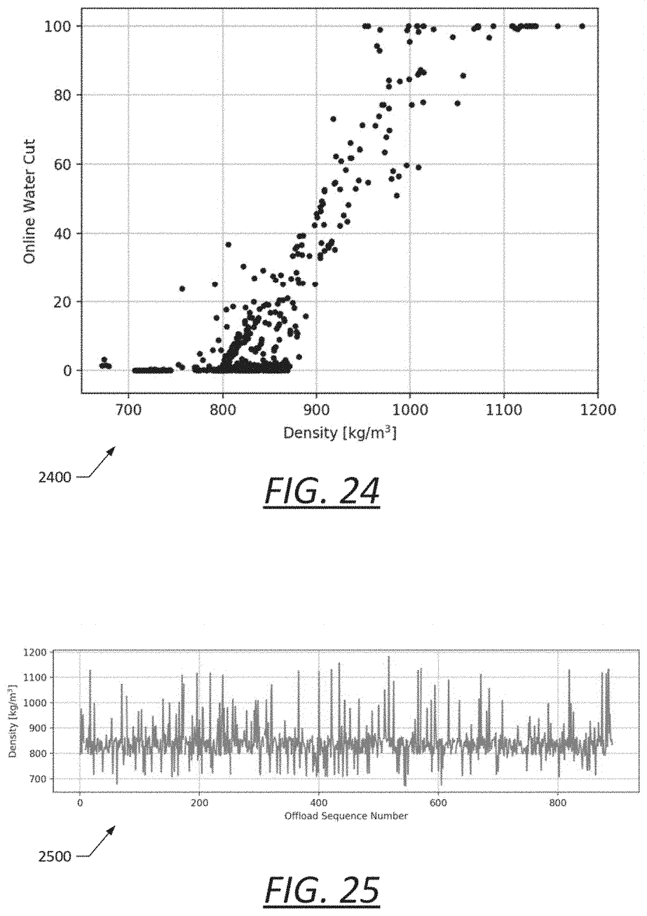

FIG. 24 shows a chart used in failure mode analysis. The chart (2400) may be generated by the analysis generator (1932) of the analysis server (1910) and displayed by the application (1926) on the client device (1912). The chart (2400) shows a graph of a set of points that identify an online water cut (percentage of water in the offload) as a function of the density of the offload in kilograms per cubic meter (kg/m). The chart (2400) shows that the water cut increases linearly as the density increases in the range from about 800 kg/m³ to about 1100 kg/m³.

FIG. 25 shows a chart used in failure mode analysis. The chart (2500) may be generated by the analysis generator (1932) of the analysis server (1910) and displayed by the application (1926) on the client device (1912). The chart (2500) shows a graph of the density of all offloads in sequence from which is shown the variability of density between offloads.

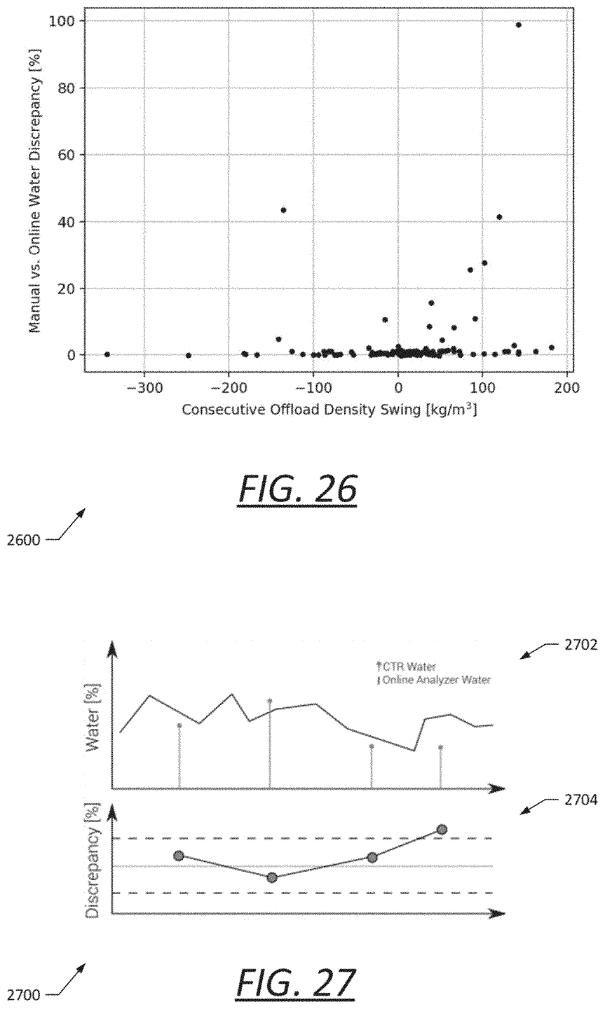

FIG. 26 shows a chart used in failure mode analysis. The chart (2600) may be generated by the analysis generator (1932) of the analysis server (1910) and displayed by the application (1926) on the client device (1912). The chart (2500) shows a graph that displays the relationship between consecutive density swings and the observed error between the online analyzers and manual water cuts. With the consecutive offload density change as an independent correlative variable, the importance of contamination (from exceptionally dense samples) and fouling errors can be diagnosed.

FIG. 27 shows a set of charts used in failure mode analysis. The set of charts (2700) may be generated by the analysis generator (1932) of the analysis server (1910) and displayed by the application (1926) on the client device (1912). The chart (2702) shows a graph that displays the calculated water cut as a function of time as derived by the measurement device (1906), which is labeled as “CTR Water”, and derived by the analyzer device (1908), which is labeled as “Online Analyzer Water”. The chart (2704) shows a graph that displays the discrepancy percentages with interpolations over time. The discrepancy percentage is the percent difference between the water cut derived by the measurement device (1906) and the water cut derived by the analyzer device (1908).

Embodiments of the invention may be implemented on a computing system. Any combination of mobile, tablet, desktop, server, router, switch, embedded device, or other types of hardware may be used. For example, as shown in FIG. 28A, the computing system (2800) may include one or more computer processors (2802), non-persistent storage (2804) (e.g., volatile memory, such as random access memory (RAM), cache memory), persistent storage (2806) (e.g., a hard disk, an optical drive such as a compact disk (CD) drive or digital versatile disk (DVD) drive, a flash memory, etc.), a communication interface (2812) (e.g., Bluetooth interface, infrared interface, network interface, optical interface, etc.), and numerous other elements and functionalities.

The computer processor(s) (2802) may be an integrated circuit for processing instructions. For example, the computer processor(s) may be one or more cores or micro-cores of a processor. The computing system (2800) may also include one or more input devices (2810), such as a touchscreen, keyboard, mouse, microphone, touchpad, electronic pen, or any other type of input device.

The communication interface (2812) may include an integrated circuit for connecting the computing system (2800) to a network (not shown) (e.g., a local area network (LAN), a wide area network (WAN) such as the Internet, mobile network, or any other type of network) and/or to another device, such as another computing device.

Further, the computing system (2800) may include one or more output devices (2808), such as a screen (e.g., a liquid crystal display (LCD), a plasma display, touchscreen, cathode ray tube (CRT) monitor, projector, or other display device), a printer, external storage, or any other output device. One or more of the output devices may be the same or different from the input device(s). The input and output device(s) may be locally or remotely connected to the computer processor(s)(2802), non-persistent storage (2804), and persistent storage (2806). Many different types of computing systems exist, and the aforementioned input and output device(s) may take other forms.

Software instructions in the form of computer readable program code to perform embodiments of the invention may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that, when executed by a processor(s), is configured to perform one or more embodiments of the invention.

The computing system (2800) in FIG. 28A may be connected to or be a part of a network. For example, as shown in FIG. 28B, the network (2820) may include multiple nodes (e.g., node X (2822), node Y (2824)). Each node may correspond to a computing system, such as the computing system shown in FIG. 28A, or a group of nodes combined may correspond to the computing system shown in FIG. 28A. By way of an example, embodiments of the invention may be implemented on a node of a distributed system that is connected to other nodes. By way of another example, embodiments of the invention may be implemented on a distributed computing system having multiple nodes, where each portion of the invention may be located on a different node within the distributed computing system. Further, one or more elements of the aforementioned computing system (2800) may be located at a remote location and connected to the other elements over a network.

Although not shown in FIG. 28B, the node may correspond to a blade in a server chassis that is connected to other nodes via a backplane. By way of another example, the node may correspond to a server in a data center. By way of another example, the node may correspond to a computer processor or micro-core of a computer processor with shared memory and/or resources.

The nodes (e.g., node X (2822), node Y (2824)) in the network (2820) may be configured to provide services for a client device (2826). For example, the nodes may be part of a cloud computing system. The nodes may include functionality to receive requests from the client device (2826) and transmit responses to the client device (2826). The client device (2826) may be a computing system, such as the computing system shown in FIG. 28A. Further, the client device (2826) may include and/or perform all or a portion of one or more embodiments of the invention.

The computing system or group of computing systems described in FIGS. 28A and 28B may include functionality to perform a variety of operations disclosed herein. For example, the computing system(s) may perform communication between processes on the same or different system. A variety of mechanisms, employing some form of active or passive communication, may facilitate the exchange of data between processes on the same device. Examples representative of these inter-process communications include, but are not limited to, the implementation of a file, a signal, a socket, a message queue, a pipeline, a semaphore, shared memory, message passing, and a memory-mapped file. Further details pertaining to a couple of these non-limiting examples are provided below.

Based on the client-server networking model, sockets may serve as interfaces or communication channel end-points enabling bidirectional data transfer between processes on the same device. Foremost, following the client-server networking model, a server process (e.g., a process that provides data) may create a first socket object. Next, the server process binds the first socket object, thereby associating the first socket object with a unique name and/or address. After creating and binding the first socket object, the server process then waits and listens for incoming connection requests from one or more client processes (e.g., processes that seek data). At this point, when a client process wishes to obtain data from a server process, the client process starts by creating a second socket object. The client process then proceeds to generate a connection request that includes at least the second socket object and the unique name and/or address associated with the first socket object. The client process then transmits the connection request to the server process. Depending on availability, the server process may accept the connection request, establishing a communication channel with the client process, or the server process, busy in handling other operations, may queue the connection request in a buffer until server process is ready. An established connection informs the client process that communications may commence. In response, the client process may generate a data request specifying the data that the client process wishes to obtain. The data request is subsequently transmitted to the server process. Upon receiving the data request, the server process analyzes the request and gathers the requested data. Finally, the server process then generates a reply including at least the requested data and transmits the reply to the client process. The data may be transferred, more commonly, as datagrams or a stream of characters (e.g., bytes).

Shared memory refers to the allocation of virtual memory space in order to substantiate a mechanism for which data may be communicated and/or accessed by multiple processes. In implementing shared memory, an initializing process first creates a shareable segment in persistent or non-persistent storage. Post creation, the initializing process then mounts the shareable segment, subsequently mapping the shareable segment into the address space associated with the initializing process. Following the mounting, the initializing process proceeds to identify and grant access permission to one or more authorized processes that may also write and read data to and from the shareable segment. Changes made to the data in the shareable segment by one process may immediately affect other processes, which are also linked to the shareable segment. Further, when one of the authorized processes accesses the shareable segment, the shareable segment maps to the address space of that authorized process. Often, only one authorized process may mount the shareable segment, other than the initializing process, at any given time.

Other techniques may be used to share data, such as the various data described in the present application, between processes without departing from the scope of the invention. The processes may be part of the same or different application and may execute on the same or different computing system.

Rather than or in addition to sharing data between processes, the computing system performing one or more embodiments of the invention may include functionality to receive data from a user. For example, in one or more embodiments, a user may submit data via a graphical user interface (GUI) on the user device. Data may be submitted via the graphical user interface by a user selecting one or more graphical user interface widgets or inserting text and other data into graphical user interface widgets using a touchpad, a keyboard, a mouse, or any other input device. In response to selecting a particular item, information regarding the particular item may be obtained from persistent or non-persistent storage by the computer processor. Upon selection of the item by the user, the contents of the obtained data regarding the particular item may be displayed on the user device in response to the user's selection.

By way of another example, a request to obtain data regarding the particular item may be sent to a server operatively connected to the user device through a network. For example, the user may select a uniform resource locator (URL) link within a web client of the user device, thereby initiating a Hypertext Transfer Protocol (HTTP) or other protocol request being sent to the network host associated with the URL. In response to the request, the server may extract the data regarding the particular selected item and send the data to the device that initiated the request. Once the user device has received the data regarding the particular item, the contents of the received data regarding the particular item may be displayed on the user device in response to the user's selection. Further to the above example, the data received from the server after selecting the URL link may provide a web page in Hyper Text Markup Language (HTML) that may be rendered by the web client and displayed on the user device.

Once data is obtained, such as by using techniques described above or from storage, the computing system, in performing one or more embodiments of the invention, may extract one or more data items from the obtained data. For example, the extraction may be performed as follows by the computing system in FIG. 28A. First, the organizing pattern (e.g., grammar, schema, layout) of the data is determined, which may be based on one or more of the following: position (e.g., bit or column position, Nth token in a data stream, etc.), attribute (where the attribute is associated with one or more values), or a hierarchical/tree structure (consisting of layers of nodes at different levels of detail-such as in nested packet headers or nested document sections). Then, the raw, unprocessed stream of data symbols is parsed, in the context of the organizing pattern, into a stream (or layered structure) of tokens (where each token may have an associated token “type”).

Next, extraction criteria are used to extract one or more data items from the token stream or structure, where the extraction criteria are processed according to the organizing pattern to extract one or more tokens (or nodes from a layered structure). For position-based data, the token(s) at the position(s) identified by the extraction criteria are extracted. For attribute/value-based data, the token(s) and/or node(s) associated with the attribute(s) satisfying the extraction criteria are extracted. For hierarchical/layered data, the token(s) associated with the node(s) matching the extraction criteria are extracted. The extraction criteria may be as simple as an identifier string or may be a query presented to a structured data repository (where the data repository may be organized according to a database schema or data format, which may be in accordance with the extensible markup language (XML) standard).

The extracted data may be used for further processing by the computing system. For example, the computing system of FIG. 28A, while performing one or more embodiments of the invention, may perform data comparison. Data comparison may be used to compare two or more data values (e.g., A, B). For example, one or more embodiments may determine any combination of A>B, A=B, A!=B, A<B, etc. The comparison may be performed by submitting A, B, and an opcode specifying an operation related to the comparison into an arithmetic logic unit (ALU) (i.e., circuitry that performs arithmetic and/or bitwise logical operations on the two data values). The ALU outputs the numerical result of the operation and/or one or more status flags related to the numerical result. For example, the status flags may indicate whether the numerical result is a positive number, a negative number, zero, etc. By selecting the proper opcode and then reading the numerical results and/or status flags, the comparison may be executed. For example, in order to determine if A>B, B may be subtracted from A (i.e., A−B), and the status flags may be read to determine if the result is positive (i.e., if A>B, then A−B>0). In one or more embodiments, B may be considered a threshold, and A is deemed to satisfy the threshold if A=B or if A>B, as determined using the ALU. In one or more embodiments of the invention, A and B may be vectors, and comparing A with B requires comparing the first element of vector A with the first element of vector B, the second element of vector A with the second element of vector B, etc. In one or more embodiments, if A and B are strings, the binary values of the strings may be compared.

The computing system in FIG. 28A may implement and/or be connected to a data repository. For example, one type of data repository is a database. A database is a collection of information configured for ease of data retrieval, modification, re-organization, and deletion. Database Management System (DBMS) is a software application that provides an interface for users to define, create, query, update, or administer databases.

The user, or software application, may submit a statement or query into the DBMS. Then the DBMS interprets the statement. The statement may be a select statement to request information, update statement, create statement, delete statement, etc. Moreover, the statement may include parameters that specify data, or data container (database, table, record, column, view, etc.), identifier(s), conditions (comparison operators), functions (e.g. join, full join, count, average, etc.), sort (e.g. ascending, descending), or others. The DBMS may execute the statement. For example, the DBMS may access a memory buffer, a reference or index a file for read, write, deletion, or any combination thereof, for responding to the statement. The DBMS may load the data from persistent or non-persistent storage and perform computations to respond to the query. The DBMS may return the result(s) to the user or software application.

The computing system of FIG. 28A may include functionality to present raw and/or processed data, such as results of comparisons and other processing. For example, presenting data may be accomplished through various presenting methods. Specifically, data may be presented through a user interface provided by a computing device. The user interface may include a GUI that displays information on a display device, such as a computer monitor or a touchscreen on a handheld computer device. The GUI may include various widgets and elements that organize what data is shown as well as how data is presented to a user. Furthermore, the GUI may present data directly to the user, e.g., data presented as actual data values through text, or rendered by the computing device into a visual representation of the data, such as through visualizing a data model.

For example, a GUI may first obtain a notification from a software application requesting that a particular data object be presented within the GUI. Next, the GUI may determine a data object type associated with the particular data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the particular data object and render a visual representation of the data values within a display device according to the designated rules for that data object type.

Data may also be presented through various audio methods. In particular, data may be rendered into an audio format and presented as sound through one or more speakers operably connected to a computing device.

Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

The above description of functions presents only a few examples of functions performed by the computing system of FIG. 28A and the nodes and/or client device in FIG. 28B. Other functions may be performed using one or more embodiments of the invention.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method for testing a test sample, comprising:
obtaining a first image of the test sample via a first input device, wherein the first input device is a primary camera configured to capture the first image while a plurality of light sources illuminate the test sample;
sending the first image from the first input device to a control panel, wherein the control panel labels a plurality of layers on the first image; and
determining a water cut of the test sample based on labeling of plurality of layers of the first image.

2. The method of claim 1, further comprising:
obtaining a second image of the test sample via a second input device;
labeling a plurality of layers on the second image of the test sample; and
averaging labeling of the first image and labeling of the second image to determine the water cut of the test sample.

3. The method of claim 1, further comprising:
obtaining a second image of the test sample via a second input device, wherein the second input device captures the second image while an ultraviolet (UV) light source of the plurality of light sources illuminates the test sample;
labeling a plurality of layers on the second image of the test sample; and
averaging labeling of the first image and the second image of the test sample to determine the water cut of the test sample.

4. The method of claim 1, further comprising:
identifying a meniscus of the test sample;
identifying a location of tick marks located on a container holding the test sample; and
calculating a volume using on the meniscus of the test sample and the tick marks located on the container.

5. The method of claim 1, further comprising:
determining a shape of a meniscus of the test sample using the first image;
determining a variation of color in the test sample using the first image; and
determining a transparency of the test sample using the first image, wherein the transparency of the test sample is taken from a lower portion of the test sample.

6. The method of claim 5, further comprising:
determining a water volume percentage based on the transparency, the variation of color, and the shape of the meniscus of the test sample;
determining a water and sediment volume percentage based on the transparency, the variation of color, and the shape of the meniscus of the test sample; and
determining a water, sediment, and interface volume percentage based on the transparency, the variation of color, and the shape of the meniscus of the test sample.

7. A method comprising:
receiving a set of timing data;
receiving a set of measurement data;
receiving a set of analyzer data;
generating a set of outputs from the timing data, the measurement data, and the analyzer data;
generating an alert from an output of the set of outputs; and
sending the output and the alert to a client device.

8. The method of claim 7, wherein generating the set of outputs comprises:
obtaining a set of images and an ultraviolet image from a measurement device in the measurement data;
for each image of the set of images:
identifying a meniscus;
classifying a layer;
computing a volume of the layer;
compositing the set of images into averaged image by averaging values of the images; and
combining the averaged image with the ultraviolet image.

9. The method of claim 7, wherein generating the set of outputs comprises:
generating a probability distribution function using a set of error functions based on historical data and a set of probability distribution functions based on real time data with a Bayesian inference based model; and
generating a recommendation by checking the probability distribution function against a set of error models.

10. The method of claim 7, wherein generating the set of outputs comprises:
extracting a set of features from the measurement data,
wherein the measurement data includes a set of images, and
wherein the set of features includes a color, transparency, an ultraviolet absorbance, and a meniscus shape that are derived from the set of images;
generating a first probability distribution function from the set of features using a naive Bayes classifier; and
generating a second probability distribution function by combining the first probability distribution function with a set of probability distribution functions and a set of weights.

11. A system, comprising:
a computer processor;
a memory;
a set of instructions in the memory that when executed by the computer processor cause the computer processor to perform the steps of:
obtaining a first image of the test sample via a first input device, wherein the first input device is a primary camera configured to capture the first image while a plurality of light sources illuminate the test sample;
sending the first image from the first input device to a control panel, wherein the control panel labels a plurality of layers on the first image; and
determining a water cut of the test sample based on labeling of plurality of layers of the first image.

12. The system of claim 11, wherein the set of instructions further cause the processor to perform the steps of:
obtaining a second image of the test sample via a second input device;
labeling a plurality of layers on the second image of the test sample; and
averaging labeling of the first image and labeling of the second image to determine the water cut of the test sample.

13. The system of claim 11, wherein the set of instructions further cause the processor to perform the steps of:
obtaining a second image of the test sample via a second input device, wherein the second input device captures the second image while an ultraviolet (UV) light source of the plurality of light sources illuminates the test sample;
labeling a plurality of layers on the second image of the test sample; and
averaging labeling of the first image and the second image of the test sample to determine the water cut of the test sample.

14. The system of claim 11, wherein the set of instructions further cause the processor to perform the steps of:
identifying a meniscus of the test sample;
identifying a location of tick marks located on a container holding the test sample; and
calculating a volume using on the meniscus of the test sample and the tick marks located on the container.

15. The system of claim 11, wherein the set of instructions further cause the processor to perform the steps of:
determining a shape of a meniscus of the test sample using the first image;
determining a variation of color in the test sample using the first image; and
determining a transparency of the test sample using the first image, wherein the transparency of the test sample is taken from a lower portion of the test sample.

16. The system of claim 15, wherein the set of instructions further cause the processor to perform the steps of:
determining a water volume percentage based on the transparency, the variation of color, and the shape of the meniscus of the test sample;
determining a water and sediment volume percentage based on the transparency, the variation of color, and the shape of the meniscus of the test sample; and
determining a water, sediment, and interface volume percentage based on the transparency, the variation of color, and the shape of the meniscus of the test sample.

17. A non-transitory computer readable medium, the non-transitory computer readable medium comprising computer readable program code for:
receiving a set of timing data;
receiving a set of measurement data;
receiving a set of analyzer data;
generating a set of outputs from the timing data, the measurement data, and the analyzer data;
generating an alert from an output of the set of outputs; and
sending the output and the alert to a client device.

18. The non-transitory computer readable medium of claim 17, wherein the computer readable program code for generating the set of outputs further comprises computer readable program code for:
obtaining a set of images and an ultraviolet image from a measurement device in the measurement data;
for each image of the set of images:
identifying a meniscus;
classifying a layer;
computing a volume of the layer;
compositing the set of images into averaged image by averaging values of the images; and
combining the averaged image with the ultraviolet image.

19. The non-transitory computer readable medium of claim 17, wherein the computer readable program code for generating the set of outputs further comprises computer readable program code for:
generating a probability distribution function using a set of error functions based on historical data and a set of probability distribution functions based on real time data with a Bayesian inference based model; and
generating a recommendation by checking the probability distribution function against a set of error models.

20. The non-transitory computer readable medium of claim 17, wherein the computer readable program code for generating the set of outputs further comprises computer readable program code for:
extracting a set of features from the measurement data,
wherein the measurement data includes a set of images, and
wherein the set of features includes a color, transparency, an ultraviolet absorbance, and a meniscus shape that are derived from the set of images;
generating a first probability distribution function from the set of features using a naive Bayes classifier; and
generating a second probability distribution function by combining the first probability distribution function with a set of probability distribution functions and a set of weights.
